(19) United States
(12) Patent Application Publication
(10) Pub. No.: US 2005/0038351 A1
(43) Pub. Date: Feb. 17, 2005

(54) Method and system for evaluating cardiac ischemia based on heart rate fluctuations

(76) Inventors: Joseph M. Starobin, Greensboro, NC (US); Yuri B. Chernyak, Waltham, MA (US)

Correspondence Address: MYERS BIGEL SIBLEY & SAJOVEC, PO BOX 37428, RALEIGH, NC, 27627, US

(21) Appl. No.: 10/625,133

(22) Filed: Jul. 23, 2003

(51) Int. Cl. A61B 5/0402

(52) U.S. Cl. 600/516

(57) ABSTRACT

A method of assessing cardiac ischemia in a subject to provide a measure of cardiovascular health in that subject is described. In general, the method comprises the steps of: (a) collecting a first RR-interval data set from the subject during a stage of gradually increasing heart rate; (b) collecting a second RR-interval data set from the subject during a stage of gradually decreasing heart rate (e.g., after an abrupt stop in exercise; during a stage of gradually decreasing exercise load; etc.); (c) separating fluctuations from a slow trend in the first RR-interval data set; (d) separating fluctuations from a slow trend in the second RR-interval data set; (e) comparing the fluctuations of the first RR-interval data set to the fluctuations of the second RR-interval data set to determine a difference between the fluctuation data sets; and (f) generating from the comparison of step (e) a measure of cardiac ischemia during stimulation in the subject, wherein a greater difference between the first and second data sets indicates greater cardiac ischemia and lesser cardiac or cardiovascular health in the subject.

### FIELD OF THE INVENTION

The present invention relates to non-invasive high-resolution diagnostics of cardiac ischemia based on the processing of heart rate data collected via either body-surface electrocardiogram (ECG) or other pulse or blood pressure measuring devices. A quantitative measure of cardiac ischemia provided by the invention may simultaneously characterize both cardiac health itself and cardiovascular system health in general.

### BACKGROUND OF THE INVENTION

Heart attacks and other ischemic events of the heart are among the leading causes of death and disability in the United States. In general, the susceptibility of a particular patient to heart attack or the like can be assessed by examining the heart for evidence of ischemia (insufficient blood flow to the heart tissue itself resulting in an insufficient oxygen supply) during periods of elevated heart activity. Of course, it is highly desirable that the measuring technique be sufficiently benign to be carried out without undue stress to the heart (the condition of which might not yet be known) and without undue discomfort to the patient.

The cardiovascular system responds to changes in physiological stress by adjusting the heart rate, which can be evaluated by measuring the time between consecutive heartbeats. This can be done either electro-cardiographically, for example by measuring time intervals between similar consecutive waves on the surface ECG, such as R waves that represent occurrence of consecutive heartbeats, or by any appropriate means for detecting the timing of each heartbeat (FIG. 1).

Recent advances in computer technology have led to improvements in automatic analysis of the heart rate and QT interval variability. It is well known that QT interval variability (dispersion) observations performed separately or in combination with heart rate (or RR-interval) variability analysis provides an effective tool for the assessment of individual susceptibility to cardiac arrhythmias (B. Surawicz, J. Cardiovasc. Electrophysiol, 1996, 7, 777-784). Applications of different types of QT and some other interval variability to susceptibility to cardiac arrhythmias are described in U.S. patents by Chamoun U.S. Pat. No. 5,020,540, 1991; Wang No. U.S. Pat. No. 4,870,974, 1989; Kroll et al. U.S. Pat. No. 5,117,834, 1992; Henkin et al. No. 5,323,783, 1994; Xue et al. U.S. Pat. No. 5,792,065, 1998; Lander U.S. Pat. No. 5,827,195, 1998; Lander et al. U.S. Pat. No. 5,891,047, 1999; Hojum et al. U.S. Pat. No. 5,951,484, 1999).

It was recently found that cardiac electrical instability can be also predicted by linking the QT—dispersion observations with the ECG T-wave alternation analysis (Verrier et al., U.S. Pat. Nos. 5,560,370; 5,842,997; 5,921,940). This approach is somewhat useful in identifying and managing individuals at risk for sudden cardiac death. The authors report that QT interval dispersion is linked with risk for arrhythmias in patients with long QT syndrome. However, QT interval dispersion alone, without simultaneous measurement of T-wave alternation, is said to be a less accurate predictor of cardiac electrical instability (U.S. Pat. No. 5,560,370 at column 6, lines 4-15).

Another application of the QT interval dispersion analysis for prediction of sudden cardiac death is described by J. Sarma (U.S. Pat. No. 5,419,338). He describes a method of an autonomic nervous system testing that is designed to evaluate the imbalances between both parasympathetic and sympathetic controls on the heart and, thus, to indicate a predisposition for sudden cardiac death.

The same author suggested that an autonomic nervous system testing procedure might be designed on the basis of the QT hysteresis (J. Sarma et al., PACE 10, 485-491 (1988)). Hysteresis between exercise and recovery was observed, and was attributed to sympatho-adrenal activity in the early post-exercise period. Such an activity was revealed in the course of QT interval adaptation to changes in the RR interval during exercise with rapid variation of the load.

The influence of sympatho-adrenal activity and the sharp dependence of this hysteresis on the time course of abrupt QT interval adaptation to rapid changes in the RR interval dynamics radically overshadows the method's susceptibility to the real ischemic-like changes of cardiac muscle electrical parameters and cardiac electrical conduction. Therefore, this type of hysteresis phenomenon would not be useful in assessing the health of the cardiac muscle itself, or in assessing cardiac ischemia.

A similar sympatho-adrenal imbalance-type hysteresis phenomenon was observed by A. Krahn et al. (Circulation 96, 1551-1556 (1997)(see FIG. 2 therein)). The authors state that this type of QT interval hysteresis may be a marker for long-QT syndrome. However, long-QT syndrome hysteresis is a reflection of a genetic defect of intracardiac ionic channels associated with exercise or stress-induced syncope or sudden death. Therefore, similar to the example described above, although due to two different reasons, it does not involve a measure of cardiac ischemia or cardiac muscle ischemic health.

A conventional non-invasive method of assessing coronary artery diseases associated with cardiac ischemia is based on the observation of morphological changes in a surface electrocardiogram during physiological exercise (stress test). A change of the ECG morphology, such as an inversion of the T-wave, is known to be a qualitative indication of ischemia. The dynamics of the ECG ST-segments are continuously monitored while the shape and slope, as well as ST-segment elevation or depression, measured relative to an average base line, are altering in response to exercise load. A comparison of any of these changes with average values of monitored ST-segment data provides an indication of insufficient coronary blood circulation and developing ischemia. Despite a broad clinical acceptance and the availability of computerized Holter monitor-like devices for automatic ST-segment data processing, the diagnostic value of this method is limited due to its low sensitivity and low resolution. Since the approach is specifically reliable primarily for ischemic events associated with relatively high coronary artery occlusion, its widespread use often results in false positives, which in turn may lead to unnecessary and more expensive, invasive cardiac catheterization.

Relatively low sensitivity and low resolution, which are fundamental disadvantages of the conventional ST-segment depression method, are inherent in such methods being based on measuring an amplitude of a body surface ECG signal, which signal by itself does not accurately reflect changes in an individual cardiac cell's electrical parameters normally changing during an ischemic cardiac event. A body surface ECG signal is a composite determined by action potentials aroused from discharge of hundred of thousands of individual excitable cardiac cells. When electrical activity of excitable cells slightly and locally alters during the development of exercise-induced local ischemia, its electrical image in the ECG signal on the body surface is significantly overshadowed by the aggregate signal from the rest of the heart. Therefore, regardless of physiological conditions such as stress or exercise, conventional body surface ECG data processing is characterized by a relatively high threshold (lower sensitivity) of detectable ischemic morphological changes in the ECG signal. An accurate and faultless discrimination of such changes is still a challenging signal processing problem.

Accordingly, an object of the present invention is, in some embodiments, to provide a non-invasive technique for detecting and measuring cardiac ischemia in a patient.

Another object of the invention is, in some embodiments, to provide a non-invasive technique for detecting and measuring cardiac ischemia, which technique is not unduly uncomfortable or stressful for the patient.

Another object of the invention is, in some embodiments, to provide a non-invasive technique for detecting and measuring cardiac ischemia, which technique may be implemented with relatively simple equipment.

Still another object of the invention is, in some embodiments, to provide a non-invasive technique for detecting and measuring cardiac ischemia, which technique is sensitive to low levels of such ischemia.

Still another object of the invention is, in some embodiments, to provide a non-invasive technique for detecting and measuring cardiac ischemia, which technique is inexpensive, does not required highly skilled personnel and is sufficiently simple for mass screening and monitoring of population groups for the presence of such ischemia.

### SUMMARY OF THE INVENTION

The present invention overcomes the deficiencies in the conventional ST-segment analysis. Although it may still be based on the processing of a body surface ECG signal, or an alternative technique of collecting the heart rate data, it nevertheless provides a highly sensitive and high resolution method for distinguishing changes in cardiac electrical conduction associated with developing cardiac ischemia. In addition to the significant cardiac ischemic changes detectable by the conventional method, the present invention allows one to determine much smaller ischemia-induced conditions and alterations in cardiac electrical conduction. Thus, unlike a conventional ST-segment depression ischemic analysis, the method of the present invention opens up opportunities to detect low-level cardiac ischemia (undetectable via the regular ST-segment method) and also to resolve and monitor small variations of cardiac ischemia. In particular, individuals who would be considered of the same level of cardiac and cardiovascular health according to a conventional ECG evaluation (an ST-depression method), will have different measurements if compared according to the method of the present invention, and the cardiac and cardiovascular health of an individual can be quantitatively evaluated, compared and monitored by repeated applications of the method of the present invention.

Based on this discovery, the present invention provides a highly sensitive and high resolution method of assessing cardiac ischemia. This method allows one to detect comparatively small alterations of cardiac muscle electrical excitation properties that develop during even a moderate ischemic condition. For example, consider a gradual heart rate adjustment in a particular human subject in response to slow (quasi-stationary), there-and-back changes of external physiological conditions. Ideally, when a cardiac muscle is supplied by a sufficient amount of oxygen during both gradually increasing and gradually decreasing heart rate stages, the corresponding, there-and-back, quasi-stationary interval curves which result should be virtually identical.

However, if ischemia exists, even if only to a very minor extent, there will be alterations of cardiac muscle repolarization and excitation properties for the human subject with the result that one observes as a specific quasi-stationary hysteresis loop of the heart rate fluctuations (instantaneous deviations from the trend) versus heart rate trend. Unlike non-stationary QT-RR hysteresis loops in (J. Sarma et al., supra (1987); A. Krahn et al., supra (1997)), the quasi-stationary fluctuation-trend hysteresis of the present invention does not vary substantially in the course of sympatho-adrenal interval adjustment. The domains and shapes of such loops are not significantly affected by time-dependent transients rapidly decaying during a transition from one particular heart rate to another; instead, they depend primarily on ischemia-induced changes of medium parameters. The domain encompassed by such a quasi-stationary hysteresis loop and its shape represent new quantitative characteristics that indicate cardiac muscle health itself and the health of the cardiovascular system in general. Moreover, any measure of the shape and/or domain enclosed in the hysteresis loop (a measure of a set as defined in the integral theory) possesses the property that any expansion of the domain results in an increase of the measure. Any such mathematical measure can be taken as the new characteristics of cardiac health mentioned above. An arbitrary monotonic function of such a measure would still represent the same measure in another, transformed scale.

A first aspect of the present invention is a method of assessing cardiac ischemia in a subject to provide a measure of cardiovascular health in that subject. In general, the method comprises the steps of:
(a) collecting a first RR-interval data set from the subject during a stage of gradually increasing heart rate;
(b) collecting a second RR-interval data set from the subject during a stage of gradually decreasing heart rate (e.g., after an abrupt stop in exercise; during a stage of gradually decreasing exercise load; etc.);
(c) separating fluctuations from a slow trend in the first RR-interval data set;
(d) separating fluctuations from a slow trend in the second RR-interval data set;
(e) comparing the fluctuations of the first RR-interval data set to the fluctuations of the second RR-interval data set to determine a difference between the fluctuation data sets; and
(f) generating from the comparison of step (e) a measure of cardiac ischemia during stimulation in the subject, wherein a greater difference between the first and second data sets indicates greater cardiac ischemia and lesser cardiac or cardiovascular health in the subject.

In an embodiment of the foregoing, the step (c) of separating fluctuations from at least one slow trend in the first RR-interval data set includes smoothing the first RR-interval data set to determine at least one slow trend in the first RR-interval data set; and the step (c) of separating fluctuations from at least one slow trend in the second RR-interval data set includes smoothing the second RR-interval data set to determine at least one slow trend in the second RR-interval data set. In an embodiment of the foregoing, the comparing step (e) is carried out at substantially equal trend values of the RR-intervals.

In various embodiment of the foregoing, the first and second RR-interval data sets are collected without an intervening rest stage; or the first RR-interval data set is collected during a stage of increasing exercise load and the second RR-interval data set is collected after an abrupt stop of exercise; etc.

In embodiments of the foregoing, the first and second RR-interval data sets are collected under quasi-stationary conditions.

In embodiments of the foregoing, the stage of gradually increasing heart rate and the stage of gradually decreasing heart rate are each at least 3 minutes in duration.

In embodiments of the foregoing, the stage of gradually increasing heart rate and the stage of gradually decreasing heart rate are together carried out for a total time of from 6 minutes to 40 minutes.

In embodiments of the foregoing, both the stage of gradually increasing heart rate and the stage of gradually decreasing heart rate are carried out between a peak rate and a minimum rate; and the peak rates of both the stage of gradually increasing heart rate and the stage of gradually decreasing heart rate are the same.

In embodiments of the foregoing, the minimum rates of both the stage of gradually increasing heart rate and the stage of gradually decreasing heart rate are substantially the same.

In embodiments of the foregoing, the stage of gradually decreasing heart rate is carried out at at least three different heart-rate stimulation levels.

In embodiments of the foregoing, the stage of gradually increasing heart rate is carried out at at least three different heart-rate stimulation levels.

In embodiments of the foregoing, the stage of gradually increasing heart rate and the stage of gradually decreasing heart rate are carried out sequentially in time.

In embodiments of the foregoing, the stage of gradually increasing heart rate and the stage of gradually decreasing heart rate are carried out separately in time.

In some embodiments of the foregoing, the heart rate during the stage of gradually increasing heart rate does not exceed more than 120 beats per minute; in other embodiments of the foregoing, the heart rate during the stage of gradually increasing heart rate exceeds 120 beats per minute.

In embodiments of the foregoing, the first and second RR-interval data sets are collected by pulse or blood pressure monitoring.

In embodiments of the foregoing, the comparing step is preceded by the step of generating fluctuation curves for each of the data sets.

In embodiments of the foregoing, the comparing step includes comparing the shapes of the fluctuation curves of each of the data sets.

In embodiments of the foregoing, the comparing step includes determining a measure of the domain between the fluctuation curves.

In embodiments of the foregoing, the comparing step includes a step of connecting the curves with a connecting segment to form a closed domain bounded by the fluctuation curves and the connecting segment.

In embodiments of the foregoing, the comparing step includes determining a measure of the domain bounded by the fluctuation curves and the connecting segment.

In some embodiments of the foregoing, the comparing step includes both comparing the shapes of the fluctuation curves and determining a measure of the domain between the fluctuation curves.

Some embodiments of the foregoing further comprise the step of displaying the fluctuation curves.

In some embodiments of the foregoing, the separating step (c), the separating step (d) and the comparing step (e) are carried out by: (i) smoothing the first and second RR-interval data sets to generate first and second slow trend data sets; (ii) separating fluctuations from the second the slow trend in the first and second data sets to generate first and second fluctuation data sets; (iii) generating a first fluctuations versus trend curve from the first slow trend data set and the first fluctuation data set; (iv) generating a second fluctuations versus trend curve from the second slow trend data set and the second fluctuation data set; (v) generating a hysteresis loop from the first fluctuations verses trend curve and the second fluctuations versus trend curve; and (vi) determining a measure of the domain inside the smoothed hysteresis loop to thereby quantify a difference between the fluctuation data sets. Such embodiments may further comprise the step of: adding a connecting segment between the first and second fluctuations versus trend curve to generate a closed hysteresis loop bounded by the first and second fluctuations versus trend curves and the connecting segment, wherein the determining step is carried out by determining a measure of the domain inside the smoothed closed hysteresis loop.

In some embodiments of the foregoing, the separating step (c), the separating step (d), and the comparing step (e) are carried out by: (i) smoothing the first and second RR-interval data sets; (ii) generating first and second smoothed trend versus time curves from the smoothed first and second RR-interval data sets; (iii) generating first and second cardiac cycle length fluctuations versus time curves by separating fluctuations from slow trends; (iv) generating an open hysteresis loop having two branches from the first and second trend versus time curves and the first and second fluctuations versus time curves; (v) connecting the branches of the open hysteresis loop to generate a closed hysteresis loop; and then (vi) determining a measure of the domain inside the closed hysteresis loop to thereby quantify a difference between the fluctuation data sets. The generating step (iii) may optionally be followed by the step of fitting the first and second smoothed curves trend versus time curves. The generating step (iv) may optionally be followed by the step of smoothing the first and second fluctuation versus time curves.

Some embodiments of the foregoing further comprise the steps of: (g) comparing the measure of cardiac ischemia to at least one reference value; and then (h) generating from the comparison of step (e) a quantitative indicium of cardiac ischemia for the subject. Such embodiments may still further comprise the steps of (i) treating the subject with a cardiovascular therapy; and then (j) repeating steps (a) through (f) to assess the efficacy of the cardiovascular therapy, in which a decrease in the quantitative indicium from before the therapy to after the therapy indicates an improvement in cardiac health in the subject from the cardiovascular therapy. In such embodiments the cardiovascular therapy can be selected from the group consisting of aerobic exercise, muscle strength building, change in diet, nutritional supplement, weight loss, stress reduction, smoking cessation, pharmaceutical treatment, surgical treatment, and combinations thereof.

A further aspect of the present invention is a computer system for assessing cardiac ischemia in a subject to provide a measure of cardiac or cardiovascular health in that subject, the system comprising:
(a) computer hardware and/or software configured for providing a first RR-interval data set collected from the subject during a stage of gradually increasing heart rate;
(b) computer hardware and/or software for providing a second RR-interval data set from the subject during a stage of gradually decreasing heart rate;
(c) computer hardware and/or software for separating fluctuations from slow trends in the first RR-interval data set;
(d) computer hardware and/or software for separating fluctuations from slow trends in the second RR-interval data set;
(e) computer hardware and/or software for comparing the fluctuations of the first RR-interval data set to the fluctuations of the second RR-interval data set at equal trend values of the RR-interval to determine the difference between the fluctuation data sets;

(f) computer hardware and/or software for generating from the comparison of step (e) a measure of cardiac ischemia during stimulation in the subject, wherein a greater difference between the first and second data sets indicates greater cardiac ischemia and lesser cardiac or cardiovascular health in the subject. In such a system, the hardware and/or software (e) for comparing the fluctuations of the first RR-interval data set to the fluctuations of the second RR-interval data set may compare the fluctuations at substantially equal trend values of the RR-interval. Such a system may further comprise (g) computer hardware and/or software means for comparing the measure of cardiac ischemia to at least one reference value; and (h) computer hardware and/or software for generating from the comparison of step (e) a quantitative indicium of cardiac ischemia for the subject.

A further aspect of the present invention is a computer program product for assessing cardiac ischemia in a subject to provide a measure of cardiac or cardiovascular health in that subject, the computer program product comprising a computer usable storage medium having computer readable program code means embodied in the medium, the computer readable program code means comprising: (a) computer readable program code for comparing a first RR-interval fluctuation data set to a second first RR-interval fluctuation data set to determine the difference between the data sets; and (b) computer readable program code for generating from the code of (a) a measure of cardiac ischemia during stimulation in the subject, wherein a greater difference between the first and second fluctuation data sets indicates greater cardiac ischemia and lesser cardiac or cardiovascular health in the subject. In such a product the computer readable program code for comparing a first RR-interval fluctuation data set to a second RR-interval fluctuation data set may compare the fluctuations at substantially equal trend values of the RR-intervals. Such a product may further comprise (c) computer readable program code for comparing the measure of cardiac ischemia to at least one reference value; and (d) computer readable program code means for generating from (c) a quantitative indicium of cardiac ischemia for the subject.

The present invention is explained in greater detail in the drawings herein and the specification set forth below.

### BRIEF DESCRIPTION OF THE DRAWINGS

FIG. 1 is a schematic graphic representation of the action potential in cardiac muscle summed up over its volume and the induced electrocardiogram (ECG) recorded on a human body surface.

FIG. 2 is a block diagram of an apparatus for carrying out the present method.

FIG. 3 is an alternative block diagram of an apparatus for carrying out the present method.

FIG. 4 is a block diagram of the processing steps for data acquisition and analysis of the present invention.

FIG. 5 is a block diagram of the processing steps for an alternative realization of the data acquisition and analysis of the present invention.

FIG. 6 illustrates the experimental raw data and the processed data at different data processing steps ending with the RR-interval fluctuation hysteresis loops for a healthy 50 year old male (SCIM(RR)™=62).

FIG. 7 illustrates the experimental raw data and the processed data at different data processing steps ending with the RR-interval fluctuation hysteresis loops for a 58 year old healthy male (SCIM(RR)™=187).

FIG. 8 illustrates the experimental raw data and the processed data at different data processing steps ending with the RR-interval fluctuation hysteresis loops for a CAD male patient (61 year old, SCIM(RR)™=323).

FIG. 9 illustrates a typical rapid peripheral nervous system and hormonal control adjustment of the RR interval as a result of an abrupt stop in exercise (that is, an abrupt initiation of a rest stage).

FIG. 10 illustrates a typical slow (quasi-stationary) RR interval adjustment measured during gradually increasing and gradually decreasing cardiac stimulation.

### DETAILED DESCRIPTION OF THE PREFERRED EMBODIMENTS

The present invention is explained in greater detail below. This description is not intended to be a detailed catalog of all the different manners in which particular elements of the invention can be implemented, and numerous variations will be apparent to those skilled in the art based upon the instant disclosure.

As will be appreciated by one of skill in the art, certain aspects of the present invention may be embodied as a method, data processing system, or computer program product. Accordingly, certain aspects of the present invention may take the form of an entirely hardware embodiment, an entirely software embodiment, or an embodiment combining software and hardware aspects. Furthermore, certain aspects of the present invention may take the form of a computer program product on a computer-usable storage medium having computer readable program code means embodied in the medium. Any suitable computer readable medium may be utilized including, but not limited to, hard disks, CD-ROMs, optical storage devices, and magnetic storage devices.

Certain aspects of the present invention are described below with reference to flowchart illustrations of methods, apparatus (systems), and computer program products. It will be understood that each block of the flowchart illustrations, and combinations of blocks in the flowchart illustrations, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions specified in the flowchart block or blocks.

Computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks.

Computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

1. Definitions.

“A trend” on a data segment is a data set generally obtained from the raw data segment by smoothing. In a particular implementation herein a trend is assessed as the smoothest data set obtained by fitting the raw data on a data segment with a lowest degree polynomial (linear or quadratic, with the latter being used when the data set encompasses a single extremum, i.e., a minimum or a maximum). The total variation of the trend is always much smaller than the total variation of the raw data segment.

“A stationary data segment” is a data segment with a negligible variation of the trend.

“A slow trend” is a trend with a small but not negligible variation. A trend obtained under the quasi-stationary protocol (see example 7) is a slow trend. A duration of a stage during which the data incorporating a slow trend are collected must be approximately an order of magnitude (e.g., at least about ten times) longer than the average duration (˜1 minute) of the heart rate adjustment after an abrupt stop of exercise from a peak load rate (typically from 120 to 150 beat/min) to the rest rate (typically from 50 to 80 beat/min).

“A fluctuation” of an RR interval on a data segment as used herein refers to a set of zero sum deviations from an RR slow trend corresponding to this particular data segment. A traditional measure of fluctuations is the standard root-mean-square deviation (STD). A typical value of STD for RR interval fluctuations is of an order of magnitude (e.g., at least about ten times) smaller than the total variation of the RR interval trend during the entire load stage under quasi-stationary conditions.

“Cardiac ischemia” refers to a lack of or insufficient blood supply to an area of cardiac muscle. Cardiac ischemia usually occurs in the presence of arteriosclerotic occlusion of a single or a group of coronary arteries. Arteriosclerosis is a product of a lipid deposition process resulting in fibro-fatty accumulations, or plaques, which grow on the internal walls of coronary arteries. Such an occlusion compromises blood flow through the artery, which reduction then impairs oxygen supply to the surrounding tissues during increased physiological need—for instance, during increased exercise loads. In the later stages of cardiac ischemia (e.g., significant coronary artery occlusion), the blood supply may be insufficient even while the cardiac muscle is at rest. However, in its earlier stages such ischemia is reversible in a manner analogous to how the cardiac muscle is restored to normal function when the oxygen supply to it returns to a normal physiological level. Thus, ischemia that may be detected by the present invention includes episodic, chronic and acute ischemia.

“Exercise” as used herein refers to voluntary skeletal muscle activity of a subject that increases heart rate above that found at a sustained stationary resting state. Examples of exercise include, but are not limited to, cycling, rowing, weight-lifting, walking, running, stair-stepping, etc., which may be implemented on a stationary device such as a treadmill or in a non-stationary environment.

“Exercise load” or “load level” refers to the relative strenuousness of a particular exercise, with greater loads or load levels for a given exercise producing a greater heart rate in a subject. For example, load may be increased in weight-lifting by increasing the amount of weight; load may be increased in walking or running by increasing the speed and/or increasing the slope or incline of the walking or running surface; etc.

“Gradually increasing” and “gradually decreasing” an exercise load refers to exercise in which the subject is caused to perform an exercise under a plurality of different sequentially increasing or sequentially decreasing loads. The number of steps in the sequence can be infinite so the terms gradually increasing and gradually decreasing loads include continuous load increase and decrease, respectively.

“Intervening rest”. when used to refer to a stage following increased cardiac stimulation, refers to a stage of time initiated by a sufficiently abrupt decrease in heart stimulation (e.g. a sufficiently abrupt decrease in exercise load) so that it evokes a clear sympatho-adrenal response. Thus, an intervening rest stage is characterized by a rapid sympatho-adrenal adjustment (as further described in Example 8 below), and the inclusion of an intervening rest stage precludes the use of a quasi-stationary exercise (or stimulation) protocol (as further described in Example 9 below).

“Hysteresis” refers to a lagging of the physiological effect when the external conditions are changed.

“Hysteresis curves” refer to a pair of curves in which one curve reflects the response of a system to a first sequence of conditions, such as gradually increasing heart rate, and the other curve reflects the response of a system to a second sequence of conditions, such as gradually decreasing heart rate. Here both sets of conditions are essentially the same—i.e., consist of the same (or approximately the same) steps—but are passed in different order in the course of time. A “hysteresis loop” refers to a loop formed by the two contiguous curves of the pair.

“Electrocardiogram” or “ECG” refers to a continuous or sequential record (or a set of such records) of a local electrical potential field obtained from one or more locations outside the cardiac muscle. This field is generated by the combined electrical activity (action potential generation) of multiple cardiac cells. The recording electrodes may be either subcutaneously implanted or may be temporarily attached to the surface of the skin of the subject, usually in the thoracic region. An ECG record typically includes the single-lead ECG signal that represents a potential difference between any two of the recording sites including the site with a zero or ground potential.

“Pulse monitor” or “heart rate monitor” refers to a device that allows one to measure and to record the duration of each cardiac cycle during the monitored period of time. Such a device measures and records the time intervals between the instances when two consecutive cardiac cycles have identical phases.

“Quasi-stationary conditions” refer to a gradual change in the external conditions and/or the physiological response it causes that occurs much slower than any corresponding adjustment due to sympathetic/parasympathetic and hormonal control. If the representative time of the external conditions variation is denoted by τₑₓₜ, and τᵢₙₜ is a representative time of the fastest of the internal, sympathetic/parasympathetic and hormonal control, then “quasi-stationary conditions” indicates τₑₓₜ>>τᵢₙₜ (e.g., τₑₓₜ is at least about five times greater than τᵢₙₜ).

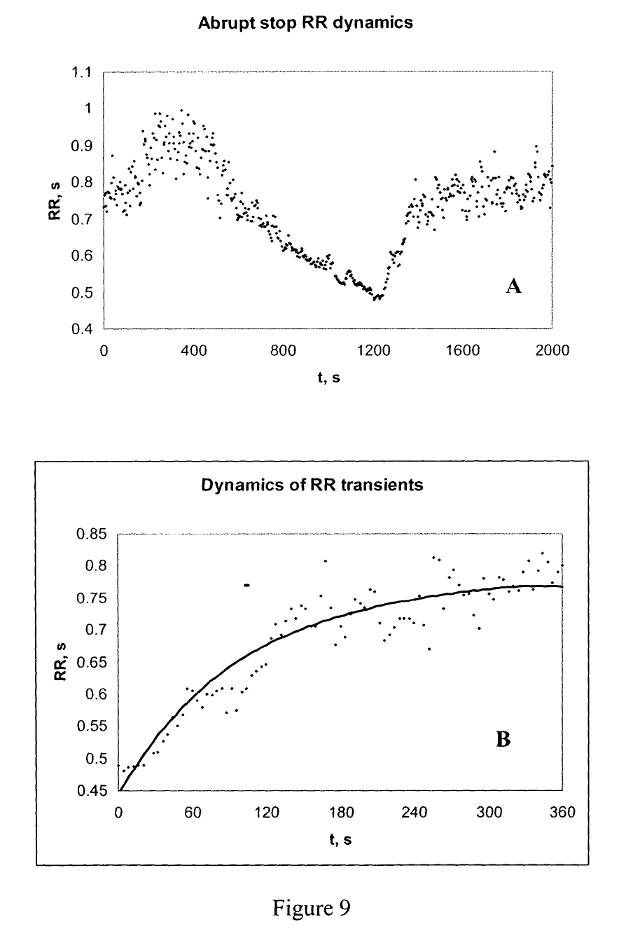

“An abrupt change” refers to an opposite situation corresponding to a sufficiently fast change in the external conditions as compared with the rate associated with sympathetic/parasympathetic and hormonal control—that is, it requires that τₑₓₜ<<τᵢₙₜ (e.g., τₑₓₜ is at least about five times less than τᵢₙₜ). In particular, “an abrupt stop” refers to a fast removal of the exercise load that occurs during time shorter than τᵢₙₜ˜20 or 30 seconds (see FIG. 9 below and comments therein).

“RR-data set” refers to a record of the time course of an electrical signal comprising action potentials spreading through cardiac muscle. Any single lead ECG record incorporates a group of three consecutive sharp deflections usually called a QRS complex and generated by the propagation of the action potential's front through the ventricles. The time interval between the cardiac cycles when observed via ECG (i.e., between the maxima of the consecutive R-waves) is called an RR-interval. Alternative definitions of these intervals can be equivalently used in the framework of the present invention. For example, an RR-interval can be defined as the time between any two similar points, such as the similar inflection points, on two consecutive R-waves, or any other way to measure cardiac cycle length. An ordered set of such interval durations simultaneously with the time instants of their beginnings or ends which are accumulated on a beat to beat basis or on any given beat sampling rate basis form an RR-interval data set. Thus, an RR-interval data set will contain two RR-interval related sequences {T_{RR,1},T_{RR,2}, . . . , T_{RR,n},} and {t₁,t₂, . . . ,tₙ}.

“Cardiac cycle length data set” refers to a record of the time course of consecutive time intervals between the instances when two consecutive cardiac cycles have identical phases. A cardiac cycle length data set can be obtained either via ECG (RR-interval data set) or the pulse or heart rate monitor. The term “cardiac cycle length data set” will be used interchangeably with the term “RR-interval data set”.

An “instantaneous heart rate” is defined as a reciprocal of a current RR interval value or, equivalently, as a reciprocal of a current cardiac cycle length.

In the following definitions, C[a,b] shall denote a set of continuous functions ƒ(t) on a segment [a,b]. {tᵢ}. i=1, 2, . . . , N, denotes a set of points from [a,b], i.e. {tᵢ}={tᵢ; a≦tᵢ≦b, i=1, 2, . . . ,N} and {ƒ(tᵢ)}, where ƒεC[a,b] denotes a set of values of the function f at the points {tᵢ}. In matrix operations the quantities τ={t₁}, y={ƒ(itᵢ)}, are treated as column vectors. E_{N} shall denote an N-dimensional metric space with the metric R_{N}(x,y), x,yεE_{N}. (R_{N}(x,y) is said to be a distance between points x and y.) A (total) variation

$${V}_{a}^{b} [ F ]$$

is defined for any absolutely continuous function F from C[a,b] as the integral (a Stieltjes integral)

$${V}_{a}^{b} [ F ( t ) ] ≡ {\int }_{a}^{b} \vert ⅆ F ( t ) \vert = {\int }_{a}^{b} \vert {F}^{′} ( t ) \vert ⅆ t . \qquad ( D .1 )$$

For a function F monotonic on segment [a,b] its variation is simply |F(a)-F(b)|. If a function F(t) has alternating maxima and minima, then the total variation of F is the sum of its variations on the intervals of monotonicity. For example, if the points of minima and maxima are x₁=a, x₂, x₃, . . . , xₖ=b, then

$${V}_{a}^{b} [ F ( t ) ] = {\sum }_{i = 1}^{k - 1} \vert F ( {x}_{i} ) - F ( {x}_{i + 1} ) \vert . \qquad ( D .2 )$$

Fitting (best fitting): Let \tilde{C}[a,b] be a subset of C[a,b]. A continuous function ƒ(t), ƒε\tilde{C} [a,b] is called the (best) fit (or the best fitting) function of class \tilde{C} [a,b] with respect to metric R_{N} to a data set {xᵢ,tᵢ{ (i=1,2, . . . , N} if

$${R}_{N} ( \{ f ( {t}_{i} ) \} , \{ {x}_{i} \} ) = {min}_{f ∈ \tilde{C} [ a , b ]} \qquad ( D .3 )$$

The minimum value of R_{N} is then called the error of the fit. The functions f(t) from \tilde{C} [a,b] will be called trial functions.

In most cases E_{N} is implied to be an Euclidean space with an Euclidean metric. The error R_{N} then becomes the familiar mean-root-square error. The fit is performed on a subset \tilde{C} [a,b] since it usually implies a specific parametrization of the trial functions and/or such constrains as the requirements that the trial functions pass through a given point and/or have a given value of the slope at a given point.

A smoother function (comparison of smoothness): Let ƒ(t) and g(t) be functions from C[a,b] that have absolutely continuous derivatives on this segment. The function ƒ(t) is smoother than the function g(t) if

$$\begin{matrix}{V}_{a}^{b} [ f ( t ) ] \leq {V}_{a}^{b} [ g ( t ) ] , & ( D .4 ) \\ and & \\ {V}_{a}^{b} [ {f}^{′} ( t ) ] \leq {V}_{a}^{b} [ {g}^{′} ( t ) ] , & ( D .5 )\end{matrix}$$

where the prime denotes a time derivative, and a strict inequality holds in at least one of relations (D.4) and (D.5).

A smoother set: A set {xᵢ,tᵢ} (i=1,2, . . . N} is smoother than the set {x′ⱼ,t′ⱼ (j=1,2, . . . , N′} if the former can be fit with a smoother function ƒ(t) of the same class within the same or smaller error than the latter.

“Smoothing” of a data set as used herein may be understood as follows: Let F and G be continuous functions on a N-dimensional space with the values in a M-dimensional space. A transformation of a data set (x,t)=≡{xᵢ,tᵢ} (i=1,2, . . . , N} into another set (y,τ)=≡{yⱼ,τⱼ} (j=1,2, . . . , M, N≧M} of the form
y=F(x). τ=G(t), (D.6)
is called a smoothing if the latter set is smoother than the former. One can refer to {yⱼ,τⱼ} as a smoothed set. In practice, the functions F and G are linear and are represented by M×N matrices (N≧M). According to the above definition a transformation of a data set by filtering (in the time or frequency domain) comprises smoothing.

A measure of a closed domain: Let Ω be a singly connected domain on the plane (τ,T) with the boundary formed by a simple (i.e., without self-intersections) continuous curve. A measure M of such a domain Ω on the plane (τ,T) is defined as the Riemann integral

$$M = {\int \int }_{Ω} ρ ( τ , T ) ⅆ τ ⅆ T \qquad ( D .7 )$$

where ρ(τ,T) is a nonnegative (weight) function on Ω.

Note that when ρ(τ,T)≡≡1 the measure M of the domain coincides with its area, A; when ρ(τ,T)≡1/τ², the measure, M, has the meaning of the area, A′, of the domain Ω′ on the transformed plane (ƒ, T), where ƒ=1/τ can be understood as the heart rate since the quantity τ has the meaning of RR-interval. [The domain Ω′ is the image of domain Ω under the mapping (τ,T)→(1/τ,T).]

“A trend” on a data segment is a data set generally obtained from the raw data segment by low pass filtering under the restriction that the deviations from the resulting trend have a zero sum. In a particular implementation herein a trend is assessed as the smoothest data set obtained by fitting the raw data on the segment with a lowest degree polynomial (linear or quadratic, with the latter being used when the data set encompasses a single extremum, i.e. a minimum or a maximum). The total variation of the trend is always much smaller than the total variation of the raw data segment.

“A stationary data segment” is a data segment with a negligible variation of the trend.

“A slow trend” is a trend with a small but not negligible variation. A trend obtained under the quasi-stationary protocol (see example 7) is a slow trend. A duration of a stage during which the data incorporating a slow trend are collected must be approximately an order of magnitude (e.g., at least about ten times) longer than the average duration (˜1 minute) of the heart rate adjustment after an abrupt stop of exercise from a peak load rate (typically from 120 to 150 beat/min) to the rest rate (typically from 50 to 80 beat/min).

“A fluctuation” of RR interval on a data segment as used herein refers to a set of zero sum deviations from an RR slow trend corresponding to this particular data segment. A traditional measure of fluctuations is the standard root-mean-square deviation (STD). A typical value of STD for RR interval fluctuations is of an order of magnitude (e.g., at least about ten times) smaller than the total variation of the RR interval trend during the entire load stage under quasi-stationary conditions.

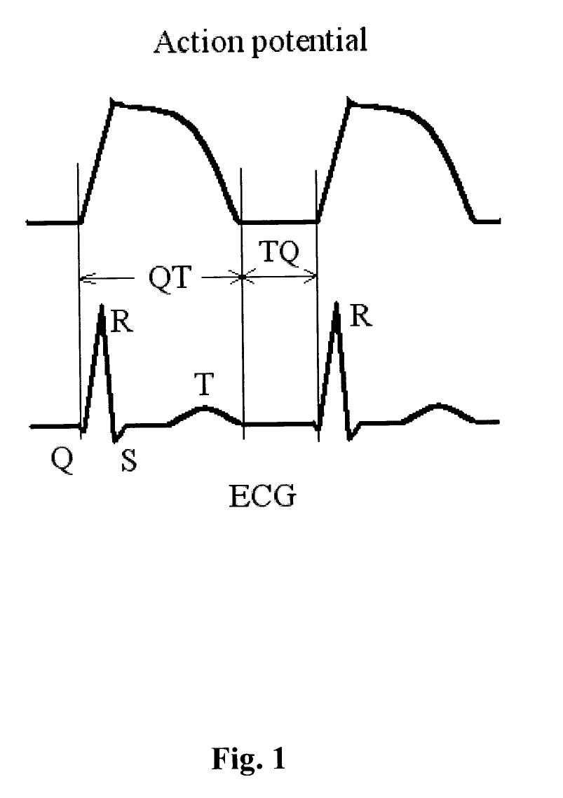

FIG. 1 illustrates the correspondence between the temporal phases of the periodic action potential (AP, upper graph, 20) generated inside cardiac muscle and summed up over its entire volume and the electrical signal produced on the body surface and recorded as an electrocardiogram (ECG, lower graph, 21). The figure depicts two regular cardiac cycles. During the upstroke of the action potential the QRS-complex is formed. It consists of three waves, Q, R, and S, which are marked on the lower panel. The recovery stage of the action potential is characterized by its fall off on the AP plot and by the T-wave on the ECG plot. One can see that the time between consecutive R-waves (RR interval) conveniently represents the duration of a cardiac cycle, while its reciprocal value represents the corresponding instantaneous heart rate.

2. Fluctuation Analysis.

Finding the mean values of measured quantities has traditionally been the center point of data processing. In live systems, however, fluctuations, the deviations from the mean value, may carry the primary information about a subsystem, which interacts with the rest of the system. In the case of cardiac stress testing, the temporal heart rate fluctuations provide additional information on the state of ventricular muscle and more generally on cardiac function during exercise. The heart rate fluctuations or the fluctuations in the cardiac cycle length can be spoken of as the RR fluctuations. The RR fluctuation time series must be extracted from the original RR interval data sets.

2.1. Theoretical basis. The idea underlying the data processing algorithms that allow one to find the temporary fluctuations of the random process with non-stationary mean value and stationary increments (first differences) has been first laid by A. Kolmogoroff (Soviet Mathematics, Doklady, 26:6-9 (1940); and 26:115-118(1940)) and later developed by Yaglom (Matematiclieskii Sbornik, 37:141-196(1955)) for the case with stationary higher order differences. Let us denote for brevity the RR interval immediately preceding a time instant t by a single quantity, T(t). Measurements of T(t) result in a discrete time series, which is a sample of the stochastic process T(t), which can be divided into two components, a nonrandom component ƒ(t) and a random component (fluctuations or physiological and physical noise) φ(t), so we have
T(t)=\overline{T}(t)+δT(t), \overline{T}(t)≡<T>, <δT(t)>=0, (2.1)
where the angle brackets denote ensemble averaging. The condition that the ensemble average of the fluctuations <δT> is zero is of crucial importance and must be preserved by any consistent data processing procedure. We shall consider sufficiently short segments of data records such that the random component, δT(t), can be considered as a stationary stochastic process with zero mean, and time independent moments. For brevity, we shall denote {T^{k}_{RR}} by {T^{k}} (k=1, 2, . . . , M). Let us consider a short segment of data {T^{k}} such that the trend can be accurately represented by a low power polynomial, e.g, a linear, or quadratic, in the vicinity of the minimum. In the former case we represent the sequence T^{k} on the segment by the expression
T^{k}=b(tₖ−tₗ)+c+δT^{k}, (2.2)
where δT^{k} by definition is the k-th fluctuation if b and c are determined by the requirement that the error E is minimized:

$$E ≡ {\sum }_{k = 1}^{M} {( δ {T}^{k} )}^{2} = {min}_{b , c} \qquad ( 2.3 )$$

This condition determines the coefficients a and b and thereby a sequence of the varying trend values, \overline{T}(tₖ)=b(tₖ−tₗ)+c, and the fluctuation time series, δT(tₖ)≡δT^{k} for k=1,2 . . . , M. In the vicinity of the HR maximum, or RR interval minimum, one needs to use a parabolic fit for the trend and set
T^{k}=a(tₖ−tₗ)²+b(tₖ−tₗ)+c+δT^{k} (2.4)
and determine the coefficients a, b and c by the similar requirement

$$E ≡ {\sum }_{k = 1}^{M} {( δ {T}^{k} )}^{2} = {min}_{a , b , c} \qquad ( 2.5 )$$

One can easily check that one of the minimization equations, ∂E/∂c=0, reduces to the requirement that

$${\sum }_{k = 1}^{M} δ {T}^{k} = 0 , \qquad ( 2.6 )$$

which, indeed, allows one to interpret the series {δT^{k}} as a series of fluctuations, a stationary random process with a zero mean value. It is also noteworthy that under the condition of quasi-stationarity the above constants a and b are sufficiently small so the trend variation of function \overline{T}(t) is much smaller than its representative value on the segment. In the linear case it means that the following condition holds
b(t_{N}−tₖ)<b(t_{N}−tₗ)<<c, (k=1, 2, . . . , M). (2.7)
Similarly, in the quadratic case it means that
a(t_{N}−tₖ)²<a(t_{N}−tₗ)²<<c, b(t_{N}−tₗ)<<c, (k=1, 2, . . . , M). (2.8)

2.2. An algorithm for finding and separating the trend and fluctuations. The above ideas can be applied to the time series within a moving, relatively short time window with the width determined by some additional requirements discussed below. Let {(tₖ,Tₖ): k=1,2, . . . N} be a set of raw data points obtained in the quasi-stationary exercise test. The set {Tₖ} is a shorthand notation for the RR-interval data set {T^{k}_{RR}} or an equivalent cardiac cycle length data set. The time instants {tₖ} are assumed to be equidistant, tₖ-tₖ₋ₗ=τₛ=const, where the time spacing τₛ is in fact determined by the beat sampling rate, which is equal to 1/τₛ. We define a k^{th} time window with a given width (2 m+1)τₛ as a set of 2m+1 points {(tⱼ,Tⱼ): j=k−m, k−m+1, . . . k+m} that include and surround point (tₖ,Tₖ). Let us denote by ƒₖ(t) a quadratic or linear polynomial obtained by a linear regression such that (t,ƒₖ(t)) provides the best fit for the data points {tⱼ,Tⱼ} within the window of a given width as defined by Equations (2.3) or (2.5) for a linear or quadratic polynomial function, ƒₖ, respectively. The function ƒₖ(t) describes the slow trend inside the window with the width M=2m+1 as. The set of corresponding fluctuations δTⱼ(m) within the window is defined by the equation
δTⱼ(m)=Tⱼ−ƒₖ(tⱼ), j=k−m,k−m+1 , . . . ,k+m. (2.9)
For a polynomial ƒₖ(t) the standard deviation (STD) for this particular window is given by the equation

$${σ}_{k}^{m} = \sqrt{\frac{1}{2 m} {\sum }_{j = k - m}^{j = k + m} {[ {T}_{j} - {f}_{k} ( {t}_{j} ) ]}^{2}} \qquad ( 2.10 )$$

The error minimization corresponding to Equations (2.3) or (2.5) is equivalent (at fixed value of m) to minimization of the STD given by Equation (2.10). This procedure is repeated for a broad range of m values. Then an optimal value of m is found by the requirement that we achieve the best accuracy (or systematic error in the trend). In fact, this requires that σₖ^{m} does not possess a clear trend as a function of the window width or a function of m. In fact, this requirement can be replaced by the condition that that the variation of σₖ^{m}/√m is minimum as a function of m.

$${σ}_{k} = {min}_{m , m > {m}_{min}} \{ \frac{{σ}_{k}^{m}}{\sqrt{m}} \} \qquad ( 2.11 )$$

The lower bound of m-values must be determined by the practical considerations such as robustness and stability of the results. Equation (2.11) defines the optimum value of m=mₒₚₜᵢₘᵤₘ. Thus optimized value of σₖ(m=mₒₚₜᵢₘᵤₘ) is taken as the current measure of fluctuations for the given, k^{th} window. The same value of m=mₒₚₜᵢₘᵤₘ also determines both the trend value within given window centered on point tₖ, which should be taken equal to the value of ƒₖ(tₖ) at the center of the window, i.e. fₖ(tₖ) As the next step we shift the center of the window one time-step further, to tₖ₊₁ and proceed to evaluation of the trend and σₖ₊₁ in the same way as at the just completed previous step. If N is the total size of the sample (number of data points) this procedure is performed N−2m times and produces N−2m values of σₖ; the respective slow trend values ƒₖ(tₖ).

3. Testing Methods.

The methods of the present invention are primarily intended for the testing of human subjects. Virtually any human subject can be tested by the methods of the present invention, including male, female, juvenile, adolescent, adult, and geriatric subjects. The methods may be carried out as an initial screening test on subjects for which no substantial previous history or record is available, or may be carried out on a repeated basis on the same subject (particularly where a comparative quantitative indicium of an individual's cardiac health over time is desired) to assess the effect or influence of intervening events and/or intervening therapy on that subject between testing sessions.

As noted above, the method of the present invention generally comprises (a) collecting a first RR-interval data set from said subject during a stage of gradually increasing heart rate; (b) collecting a second RR-interval data set from said subject during a stage of gradually decreasing heart rate; (c) determining the trend in the first cardiac cycle length data set and separating deviations from the trend (fluctuations) in the first cardiac cycle length data set; (d) determining the trend in the second cardiac cycle length data set and separating deviations from the trend (fluctuations) in the second cardiac cycle length data set; (e) comparing the fluctuations of the first cardiac cycle length data set to the fluctuations of the second cardiac cycle length data set at equal trend values of the cardiac cycle length to determine the difference between the fluctuation data sets; and (f) generating from the comparison of step (e) a measure of cardiac ischemia during stimulation in the subject, wherein a greater difference between the first and second data sets indicates greater cardiac ischemia and lesser cardiac or cardiovascular health in the subject.

The stages of gradually increasing and gradually decreasing heart rate are carried out in a manner that maintains during both periods essentially or substantially the same stimulation of the heart by the peripheral nervous and hormonal control systems, so that it is the effect of cardiac ischemia rather than that of the external control which is measured by means of the present invention. This methodology can be carried out by a variety of techniques, with the technique of conducting two consecutive stages of gradually increasing and gradually decreasing exercise loads (or average heart rates) being currently preferred.

The stage of gradually increasing exercise load (or increased average heart rate) and the stage of gradually decreasing exercise load (or decreased average heart rate) may be the same in duration or may be different in duration. In general, each stage is at least 3, 5, 8, or 10 minutes or more in duration. Together, the duration of the two stages may be from about 6, 10, 16 or 20 minutes in duration to about 30, 40, or 60 minutes in duration or more. The two stages are preferably carried out sequentially in time-that is, with one stage following after the other substantially immediately, without an intervening rest stage. In the alternative, the two stages may be carried out separately in time, with an intervening “plateau” stage (e.g., of from 1 to 5 minutes) during which cardiac stimulation or exercise load is held substantially constant, before the stage of decreasing load is initiated.

The exercise protocol may include the same or different sets of load steps during the stages of increasing or decreasing heart rates. For example, the peak load in each stage may be the same or different, and the minimum load in each stage may be the same or different. In general, each stage consists of at least two or three different load levels, in ascending or descending order depending upon the stage. Relatively high load levels, which result in relatively high heart rates, can be used but are not essential. An advantage of the present invention is that its sensitivity allows both exercise procedures to be carried out at relatively low load levels that do not unduly increase the pulse rate of the subject. For example, the method may be carried out so that the heart rate of the subject during either the ascending or descending stage (or both) does not exceed about 140, 120, or even 100 beats per minute, depending upon the condition of the subject. Of course, data collected at heart rates above 100, 120, or 140 beats per minute may also be utilized if desired, again depending upon the condition of the subject.

For example, for an athletic or trained subject, for the first or ascending stage, a first load level may be selected to require a power output of 60 to 100 or 150 watts by the subject; an intermediate load level may be selected to require a power output of 100 to 150 or 200 watts by the subject; and a third load level may be selected to require a power output of 200 to 300 or 450 watts or more by the subject. For the second or descending stage, a first load level may be selected to require a power output of 200 to 300 or 450 watts or more by the subject; an intermediate or second load level may be selected to require a power output of 100 to 150 or 200 watts by the subject; and a third load level may be selected to require a power output of 60 to 100 or 150 watts by the subject. Additional load levels may be included before, after, or between all of the foregoing load levels as desired, and adjustment between load levels can be carried out in any suitable manner, including step-wise or continuously.

In a further example, for an average subject or a subject with a history of cardiovascular disease, for the first or ascending stage, a first load level may be selected to require a power output of 40 to 75 or 100 watts by the subject; an intermediate load level may be selected to require a power output of 75 to 100 or 150 watts by the subject; and a third load level may be selected to require a power output of 125 to 200 or 300 watts or more by the subject. For the second or descending stage, a first load level may be selected to require a power output of 125 to 200 or 300 watts or more by the subject; an intermediate or second load level may be selected to require a power output of 75 to 100 or 150 watts by the subject; and a third load level may be selected to require a power output of 40 to 75 or 100 watts by the subject. As before, additional load levels may be included before, after, or between all of the foregoing load levels as desired, and adjustment between load levels can be carried out in any suitable manner, including step-wise or continuously.

The heart rate may be gradually increased and gradually decreased by subjecting the patient to a predetermined schedule of stimulation. For example, the patient may be subjected to a gradually increasing exercise load and gradually decreasing exercise load, or gradually increasing electrical or pharmacological stimulation and gradually decreasing electrical or pharmacological stimulation, according to a predetermined program or schedule. Such a predetermined schedule is without feedback of actual heart rate from the patient. In the alternative, the heart rate of the patient may be gradually increased and gradually decreased in response to actual heart rate data collected from concurrent monitoring of said patient. Such a system is a feedback system. For example, the heart rate of the patient may be monitored during the test and the exercise load (speed and/or incline, in the case of a treadmill) can be adjusted so that the heart rate varies in a prescribed way during both stages of the test. The monitoring and control of the load can be accomplished by a computer or other control system using a simple control program and an output panel connected to the control system and to the exercise device that generates an analog signal to the exercise device. One advantage of such a feedback system is that (if desired) the control system can insure that the heart rate increases substantially linearly during the first stage and decreases substantially linearly during the second stage.

The generating step (f) may be carried out by any suitable means, such as by generating curves from the data sets (with or without actually displaying the curves), and then (i) directly or indirectly evaluating a measure (e.g., as defined in the integral theory) of the domain (e.g., area) inside the hysteresis loop, a greater measure indicating greater cardiac ischemia in said subject, (ii) directly or indirectly comparing the shapes (e.g., slopes or derivatives thereof) of the curves, with a greater difference in shape indicating greater cardiac ischemia in the subject; or (iii) combinations of (i) and (ii). Specific examples are given in Example 3-5 below.

The method of the invention may further comprise the steps of (e) comparing the measure of cardiac ischemia during exercise to at least one reference value (e.g., a mean, median or mode for the quantitative indicia from a population or subpopulation of individuals) and then (f) generating from the comparison of step (e) at least one quantitative indicium of cardiovascular health for said subject. Any such quantitative indicium may be generated on a one-time basis (e.g., for assessing the likelihood that the subject is at risk to experience a future ischemia-related cardiac incident such as myocardial infarction or ventricular tachycardia), or may be generated to monitor the progress of the subject over time, either in response to a particular prescribed cardiovascular therapy or simply as an ongoing monitoring of the cardiovascular physical condition of the subject for improvement or decline (again, specific examples are given in Example 3-5 below). In such a case, steps (a) through (f) above are repeated on at least one separate occasion to assess the efficacy of the cardiovascular therapy or the progress of the subject. A decrease in the difference between said data sets from before said therapy to after said therapy, or over time, indicates an improvement in cardiac health in said subject from said cardiovascular therapy. Any suitable cardiovascular therapy can be administered, including but not limited to, aerobic exercise, muscle strength building, change in diet, nutritional supplement, weight loss, smoking cessation, stress reduction, pharmaceutical treatment (including gene therapy), surgical treatment (including both open heart and closed heart procedures such as bypass, balloon angioplasty, catheter ablation, etc.) and combinations thereof.

The therapy or therapeutic intervention may be one that is approved or one that is experimental. In the latter case, the present invention may be implemented in the context of a clinical trial of the experimental therapy, with testing being carried out before and after therapy (and/or during therapy) as an aid in determining the efficacy of the proposed therapy.

4. Testing Apparatus.

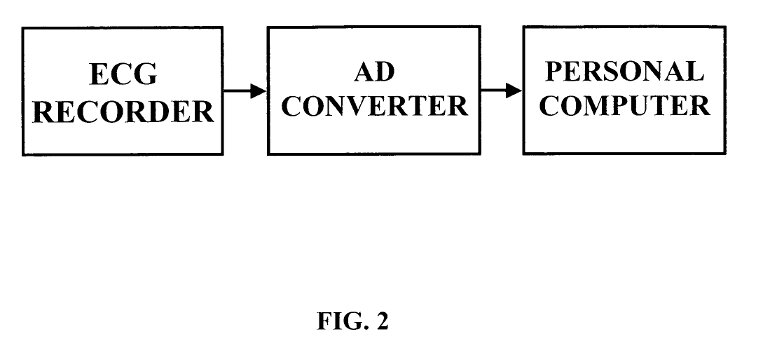

FIG. 2 provides an example of the apparatus for data acquisition, processing and analysis by the present invention. Electrocardiograms are recorded by an ECG recorder, via electrical leads placed on a subject's body. The ECG recorder may be, for example, a standard multi-lead Holter recorder or any other appropriate recorder. The analog/digital converter digitizes the signals recorded by the ECG recorder and transfers them to a personal computer, or other computer or central processing unit, through a standard external input/output port. The digitized ECG data can then be processed by standard computer-based waveform analyzer software, which identifies, in particular, R waves and their timing. The totality of such R wave timing instants translates into the RR interval data set, from which cardiac or cardiovascular health indicium or other quantitative measure of the presence, absence or degree of cardiac ischemia can then be computed automatically in the computer through a program (e.g, Basic, Fortran, C++, etc.) implemented therein as software, hardware, or both hardware and software.

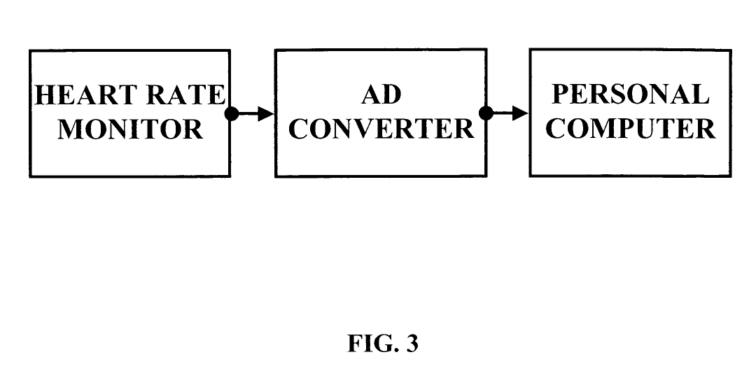

FIG. 3 provides an example of an alternative apparatus for data acquisition, processing and analysis by the present invention. The first two steps of data acquisition are performed by a Polar S810 heart rate monitor (Polar Electro Inc., 370 Crossways Park Dr., Woodbury, N.Y. 11797-2050). The actual monitor, Polar S810 heart rate monitor, incorporates an analog-digital converter so its output is directly fed to a computer in which the cardiac cycle length data set is formed and stored. Using this data set, cardiac or cardiovascular health indicium or other quantitative measure of the presence, absence or degree of cardiac ischemia can then be computed automatically in the computer through a program (e.g, Basic, Fortran, C++, etc.) implemented therein as software, hardware, or both hardware and software.

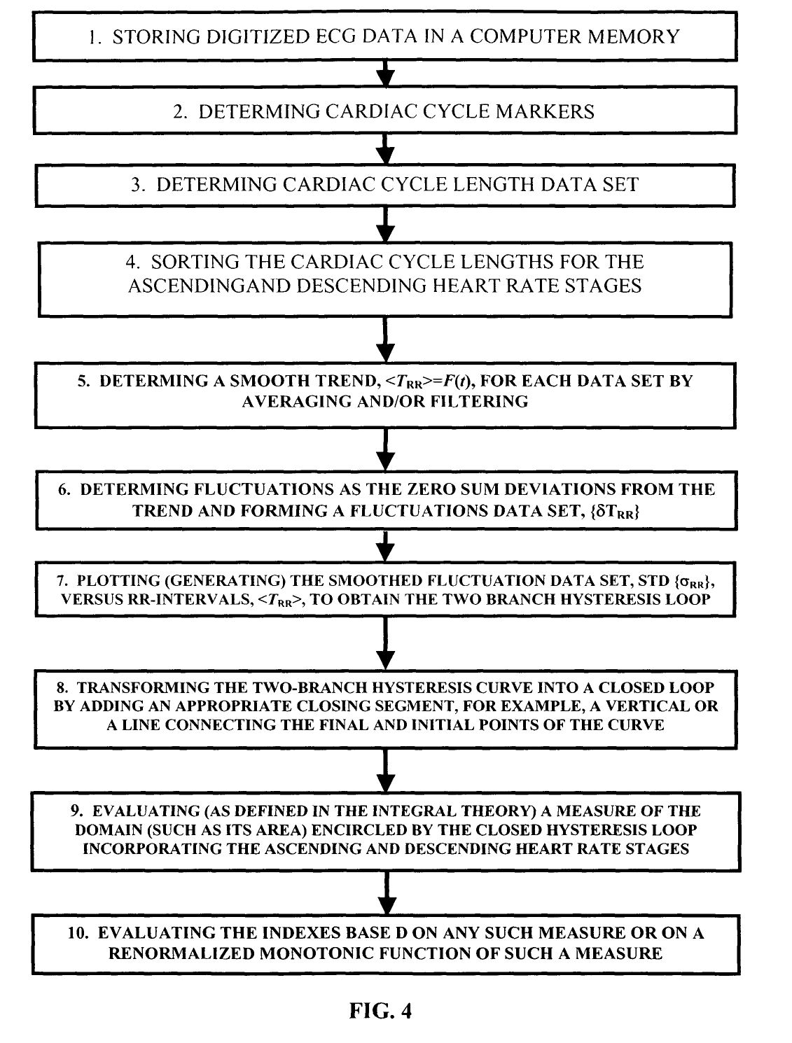

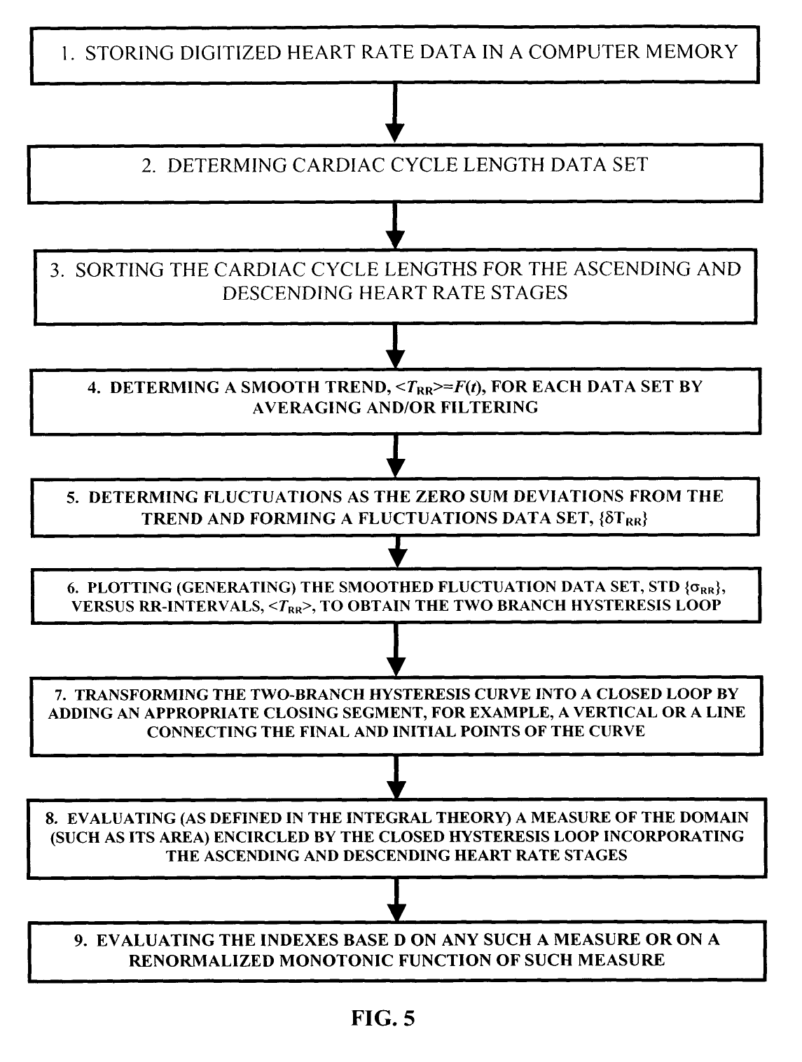

FIGS. 4 and 5 correspond to two alternative data acquisition methods represented in FIGS. 2 and 3, respectively. These figures illustrate major steps of digitized data processing involved in the analysis of an RR data set collected from a subject during there-and-back quasi-stationary changes in physiological conditions. The last seven steps in FIG. 4 and FIG. 5 are substantially the same while the initial steps differ. As shown in FIG. 4, the digitized data collected from a multi-lead recorder are stored in a computer memory for each lead as a data array (the 1^{st} step). The size of each data array is determined by the durations of the ascending and descending heart rate stages and a sampling rate used by the waveform analyzer, which processes an incoming digitized ECG signal. The waveform analyzer software first detects major characteristic waves (Q,R,S and T waves) of the ECG signal in each lead. Then in each ECG lead it determines the timing of each R wave (the 2^{nd} step). Using the data from the lead with the best data to noise ratio, the time instants of the R wave occurrence are determined as reference points to compute a set of RR intervals and a set of instantaneous heart rates (3^{rd} step).

Then, the application part of the software sorts the RR intervals for the ascending and descending heart rate stages (the 4^{th} step). The step includes computing by the application part of the software RR intervals separately for the ascending and descending heart rate stages effected by there-and-back gradual changes in physiological conditions such as exercise, pharmacological or electrical stimulation, etc. At the next, 5^{th}, step the application software performs smoothing, filtering or data fitting, using exponential or any other suitable functions, in order to obtain a sufficiently smooth trend curve T_{RR}=F(t) for each stage, including the ascending and descending heart rate stages.

At the next, 6^{th}, step the application part of the software determines fluctuations as deviations from the trend, δT_{RR}=T_{RR}−<T_{RR}> and generates a sufficiently smooth curve σ_{RR}=σ_{RR}≡\sqrt{<δT²_{RR}>}=Φ(t) to represent the standard deviations (STD) of fluctuations as a function of time during exercise.

At the 7^{th} step these parametric representations, T_{RR}=F(t) and δT_{RR}=Φ(t), are used to eliminate the time and generate or plot on the (<T_{RR}>,σ_{RR}) plane a sufficiently smooth hysteresis loop parametrically represented by the pair of functions <T_{RR}>=F(t) and σ_{RR}=Φ(t).

The next, 8^{th}, step performed by the application part of the software can be graphically presented as closing the two branch hysteresis loop with an appropriate interconnecting or partially connecting line, such as a vertical straight line or a line connecting the initial and final points, in order to produce a closed hysteresis loop on the (<T_{RR}>,σ_{RR})-plane. At the 9^{th} step the application software evaluates an appropriate measure of the domain inside the closed hysteresis loop. A measure, as defined in mathematical integral theory, is a generalization of the concept of an area and may include appropriate weight functions increasing or decreasing the contribution of different portions of the domain into said measure. The final, 10^{th}, step of the data processing is that the application software computes indexes by appropriately renormalizing the said measure or any monotonous functions of said measure. The measure itself along with the indexes may reflect both the severity of the exercise-induced ischemia, as well as a predisposition to local ischemia that can be reflected in some particularities of the shape of the hysteresis loop and the curves T_{RR}=F(t) and σ_{RR}=Φ(t). The results of all above-mentioned signal processing steps may be used to quantitatively assess cardiac ischemia and, as a simultaneous option, cardiovascular system health of a particular individual under evaluation.

Instead of using the (T_{RR},σ_{RR})-plane, a similar data processing procedure can equivalently be performed on any plane obtained by a non-degenerate transformation of the (T_{RR},σ_{RR})-plane, such as (ƒ_{RR},σ_{RR}) where ƒ_{RR}=<1/T_{RR>} is the smoothed and/or filtered heart rate or the like. Such a transformation can be partly or fully incorporated in the appropriate definition of the said measure.

FIG. 5 is similar to FIG. 4 but corresponds to the alternative data acquisition method by a pulse monitor with digital output and also represents major steps of the digitized data processing involved in the analysis of a RR data set collected from a subject during there-and-back quasi-stationary changes in physiological conditions. The data processing steps by the application software (steps 3 through 9) are substantially the same as the respective steps 4 through 10 in FIG. 4.

5. RR-Interval Monitoring with Blood Pressure and/or Pulse Signals.

A quasi-stationary RR data set can be collected non-invasively not only via measurements of a cardiac surface ECG but also by monitoring a blood pressure and/or pulse signals. In these cases, instead of the ECG recorder, a system for assessing cardiac ischemia may comprise pulse and/or blood pressure monitors, as discussed below.

A pulse monitor or pulse meter may be a suitable device, including but not limited to opto-electronic and phono or audio transducers attached to different parts of a subject's body (for example, to a finger as in finger plethysmography), if suchdevice measures a heart rate or pulse (HR). Preferably the device then computes RR-intervals equal to 1/HR and stores these data in a computer memory in order to provide further RR-interval computational analysis as described herein.

A blood pressure monitor (e.g., a sphygmomanometer) can be any suitable device, including but not limited to a cuff, a stethoscope, or an automatic pressure registering system with a digital data storage module. Many such monitoring devices are applicable even for home use and typically contain all of the modules in one unit. An automatic cuff inflation monitor may also be included in the unit. Most units are portable and have a D-ring cuff for one-handed application. The cuff usually fits around the upper arm or the wrist. These units provide personalized cuff inflation and deflation. They automatically adjust to changes in a subject's blood pressure. Blood pressure monitoring with simultaneous measurement of the HR is convenient, easy to do and takes less than a minute per measurement. As in the case above, the RR-intervals are equal to 1/HR. Such apparatus may be easily incorporated into a method and apparatus of the present invention with suitable interfaces, in accordance with known techniques.

The options described above may be used separately or in parallel, including in parallel with ECG data, depending on experimental needs. Pulse meter ischemia assessment is expected to be more accurate than blood pressure monitoring since an RR sampling frequency (frequency of HR measurements) for a pulse meter is at least an order of magnitude higher (10 to 1 data point) than in the case of blood pressure monitoring by an automatic sphygmomanometer.

6. Conditions Under Which an Abrupt Stop Exercise Protocol can be Considered as a Quasi-Stationary Protocol.

An advantage of the present invention is, however, that it can be carried out with an “abrupt stop” exercise protocol as well as a gradually increasing/gradually decreasing exercise protocol. In the present invention, an “abrupt stop” exercise protocol can be used for any individual provided that the heart rate achieved prior to the stop is sufficiently high for that individual so that his or her recovery time is sufficiently long for an appropriate hysteresis to be measured.

In general, each stage of a gradually increasing and decreasing quasi-stationary exercise protocol is at least 3, 5, 8, or 10 minutes in duration. Each stage's duration is approximately an order of magnitude longer than the average duration (˜1 minute) of heart rate (HR) adjustment during an abrupt stop of the exercise between average peak load rate (˜120-150 beat/min) and average rest (˜50-70 beat/min) heart rate values.

One should note that the ˜1 minute HR adjustment period after an abrupt stop of exercise is typical only for healthy individuals and those with not more than a relatively moderate level of coronary artery disease. However, due to rapid development of massive exercise-induced ischemia for individuals with a pronounced coronary artery disease level the same adjustment period can be significantly longer—up to 10 minutes or more in duration. In these cases the HR deviations (described in the example 9 below) are small and indicate that such an ill patient remains under a significant physical stress even after an abrupt stop of exercise, without further exercise load exposure. In such cases a physician must usually interrupt the protocol prior to completion of a full quasi-stationary exercise regimen. However, under these conditions a recovery portion of the QT/RR hysteresis loop, as well as a loop formed after an abrupt stop of exercise, can be considered as the quasi-stationary loop. Indeed, a slow heart rate recovery to a level equal to HR prior to the exercise takes 3, 5, 8, 10 or more minutes in duration with small HR deviations and, therefore, still satisfies the underlying definition of a gradual exercise protocol.

Thus, for ill patients afflicted with coronary artery disease (CAD), an abrupt stop of exercise does not prevent the completion of the method of the present invention, since due to a prolonged HR recovery stage an indicium of cardiac or cardiovascular health can still be calculated as a quasi-stationary loop index without a completion of a full exercise protocol.

Note that typically a patient with a distinguished coronary artery disease level, as determined by physical exhaustion, shortness of breath, chest pain, and/or some other clinical symptom, is unable to exercise longer than 3, 5, 8 or 10 or more minutes at even a low-level power output of about 20 watts. A gradual recovery process followed the abrupt stop of such exercise in such patients satisfies the definition of a quasi-stationary process as given herein. Note that patients with moderate levels of coronary artery disease can exercise longer, up to 20 minutes or more, and may be exposed to significantly higher exercise workouts ranging from 50 to 300 watts.

For healthy patients, an “abrupt stop” protocol can also be utilized in carrying out the present invention, for example when such patients achieve a peak level of exercise close to their maximum level of physical exertion. Under such circumstances such patients are similar to patients afflicted with CAD, who reach such a threshold under much lower exercise loads.

The present invention is explained in greater detail in the non-limiting examples set forth below.

### EXAMPLE 1

### Testing Apparatus

A testing apparatus consistent with FIG. 2 was assembled. The electrocardiograms are recorded by an RZ152PM12 Digital ECG Holter Recorder (ROZINN ELECTRONICS, INC.; 71-22 Myrtle Av., Glendale, N.Y., USA 11385-7254), via 12 electrical leads with Lead-Lok Holter/Stress Test Electrodes LL510 (LEAD-LOK, INC.; 500 Airport Way, P.O. Box L, Sandpoint, Id., USA 83864) placed on a subject's body in accordance with the manufacturer's instructions. Digital ECG data are transferred to a personal computer (Dell Dimension XPS T500 MHz/Windows 98) using a 40 MB flash card (RZFC40) with a PC 700 flash card reader, both from Rozinn Electronics, Inc. Holter for Windows (4.0.25); waveform analysis software is installed in the computer, which is used to process computer based waveform analyzer software. The hysteresis loop for each tested subject and an indicium that provides a quantitative characteristic of the extent of cardiac ischemia in said subject are then computed manually or automatically in the computer through a program implemented in Fortran 90 as illustrated in FIG. 4.

Experimental data were collected during an exercise protocol programmed in a Landice L7 Executive Treadmill (Landice Treadmills; 111 Canfield Av., Randolph, N.J. 07869). The programmed protocol included 20 step-wise intervals of a constant exercise load from 48 seconds to 1.5 minutes each in duration. Altogether these intervals formed two equal-in-duration gradually increasing and gradually decreasing exercise load stages, with total duration varying from 16 to 30 minutes. For each stage a treadmill belt speed and elevation varied there-and-back, depending on the subject's age and health conditions, from 1.5 miles per hour to 5.5 miles per hour and fi-om one to ten degrees of treadmill elevation, respectively.

### EXAMPLE 2

### An Alternative Testing Apparatus

A testing apparatus consistent with FIG. 3 was assembled. Experimental data were collected during an exercise protocol programmed in a Landice L7 Executive Treadmill in the way as described in Example 1. Instead of using a Digital ECG Holter Recorder, the instantaneous heart rate during exercise was directly measured using a Polar S810 heart rate monitor (Polar Electro Inc., 370 Crossways Park Dr., Woodbury,N.Y. 11797-2050). The Polar S810 heart rate monitor incorporates an analog-digital converter whose output is directly fed to a computer in which the data are stored as a digital array representing the cardiac cycle length data set. The hysteresis loop for each tested subject and an indicium that provides a quantitative characteristic of the extent of cardiac ischemia in said subject are then computed manually or automatically in the computer through a program implemented in Fortran 90 as illustrated in FIG. 5.

### EXAMPLES 3-5

### Human RR-fluctuation Hysteresis Studies

These examples illustrate quasi-stationary ischemia-induced RR interval fluctuation hysteresis in different human subjects. These data indicate that the method possesses a potential for high sensitivity and high resolution.

### EXAMPLE 3

### A Heart Rate Monitor Measurement of the Hysteresis Curve and SCIM™ (RR) Score in Healthy Male Subject

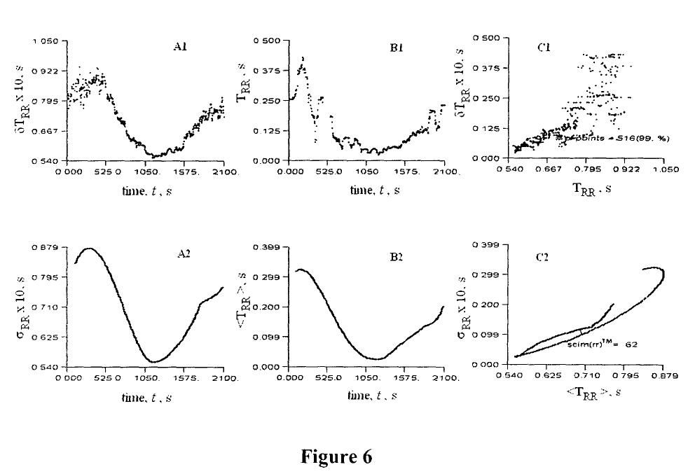

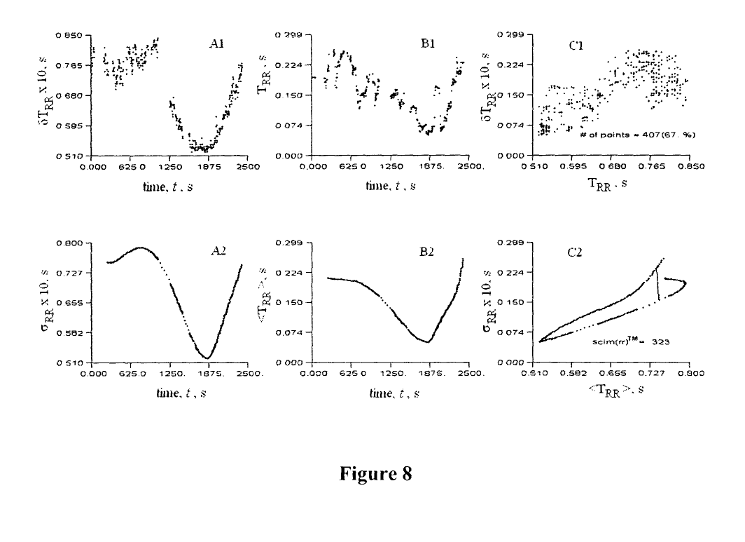

The example was carried out on a 50 year old male subject using the alternative apparatus and procedure described in Example 2 above. The subject exercised on a treadmill according to a quasi-stationary 20-minute protocol with gradually increasing and gradually decreasing exercise load. The test data obtained using alternative cardiac cycle length monitoring by a Polar S810 heart rate monitor are displayed in FIG. 3. The upper panels, A1, B1 and C1, show raw data while the lower panels, A2, B2, and C2, show smoothed and partially or fully processed data at different processing stages. Panel A1 in FIG. 6 displays cardiac cycle length data sets {T_{RR}(tⱼ)} versus time, {tⱼ}, during the exercise test. A beat sampling rate with which cardiac cycle length was sampled was equal to 12 samples per minute. Panel A2 in FIG. 6 displays filtered and smoothed data from panel A1 as a functional dependence <T_{RR}>=F(t). The smoothing procedure includes moving averaging with a two minute long moving window and is completed by the data fitting with the third-order polynomial functions within each two-minute long moving window. Panel B1 in FIG. 6 displays the raw fluctuations of the cardiac cycle length, δT_{RR}=T_{RR}-<T_{RR}> versus time during the exercise test. Panel B2 in FIG. 6 displays filtered and smoothed characterization of the fluctuation magnitude from the panel B1 as a functional dependence of the standard deviation, STD, versus time, σ_{RR}=Φ(t). Panel C1 in FIG. 6 displays the raw fluctuations versus the raw cardiac cycle length during the exercise test. Panel C2 in FIGS. 6 displays the hysteresis loop parametrically represented by the dependencies <T_{RR}>=F(t) and σ_{RR}=Φ(t) plotted in the respective panels A2 and B2. The vertical lines closing the loops (step 7 in FIG. 5) are also shown in panels C2. The subject had no conventional ischemia-induced depression of the ECG-ST segments in similar ECG stress tests under the same quasi-stationary protocol, carried out separately on multiple occasions. The area of the closed hysteresis loop is relatively small as compared with that shown in FIG. 8 and discussed in Example 5. Appropriate renormalization (see Example 7) of the area results in the RR fluctuation ischemia index SCIM™ (RR) with the value of 62. This value is significantly less than the value of SCIM™ (RR)=323 for the CAD subject considered in Example 5, below (see also FIG. 8). The fact that the method and apparatus of the present invention have precision, which is significantly higher than the difference between these two values, the method of the present invention allows one to detect and quantify ischemia with excellent resolution. This is also a clear indication of high sensitivity and specificity of the method and apparatus of the present invention.

### EXAMPLES 4

### A Holter Monitor Measurement of the Hysteresis Curve and SCIMT™ (RR) Score in Healthy Male Subject

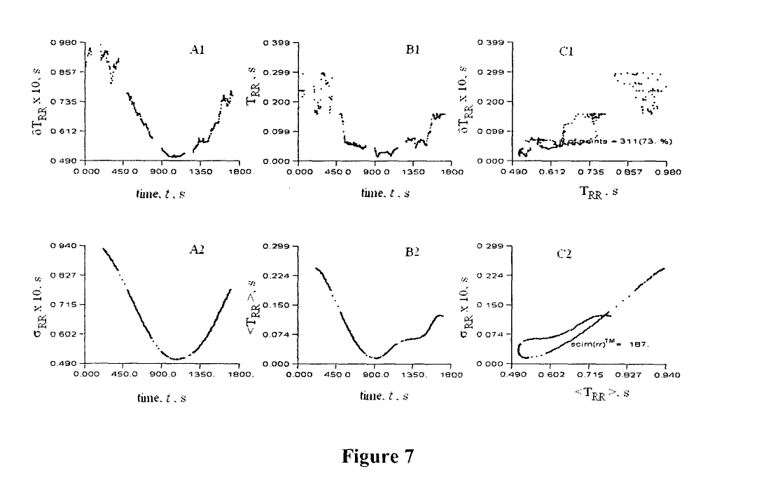

The example illustrated by FIG. 7 was carried out on a 58 years old male subject using the apparatus and procedure described in Example 2 above. The subject exercised on a treadmill according to a quasi-stationary 20-minute protocol with gradually increasing and gradually decreasing exercise load. The upper panels, A1, B1 and C1, show raw data while the lower panels, A2, B2, and C2, show partially or fully processed and smoothed data. Panel A1 in FIG. 7 displays RR interval data sets {T_{RR}(tⱼ) versus time, {tⱼ}, during the exercise test. A beat sampling rate, with which the waveform analyzer samples RR intervals was equal to 15 samples per minute. The data presented in FIG. 7 were obtained using left precordial V5 leads. Panel A2 in FIG. 7 displays filtered and smoothed data from the respective panels A1 as a functional dependence <T_{RR}>=F(t). The smoothing procedure includes moving averaging with a two minute long moving window and is completed by the data fitting with the third-order polynomial functions within each two-minute long moving window. Panel B1 in FIG. 7 displays the RR interval fluctuations, δT_{RR}=T_{RR}-<T_{RR}>, versus time, t, during the exercise test. Panel B2 in FIG. 7 displays filtered and smoothed characterization of the fluctuation magnitude from panel B1 as a functional dependence of the standard deviation, STD, versus time, σ_{RR}=Φ(t). Panel C1 in FIG. 7 displays the raw fluctuations versus raw cardiac cycle length during the exercise test. Panel C2 in FIG. 7 displays the hysteresis loop parametrically represented by the dependencies <T_{RR}>=F(t) and σ_{RR}=Φ(t) plotted in the respective panels A2 and B2. The vertical lines closing the loops (step 8 in FIG. 4) are also shown in panels C2. The ROZINN software system detected no conventional ischemia-induced depression of the ECG-ST segments during test. The areas of the closed hysteresis loop is again relatively small as compared with that shown in FIG. 8 and discussed in Example 5. Appropriate renormalization of the area (see Example 7) results in the RR fluctuation ischemia index SCIM™(RR) with the value of 187. This value is significantly less than the value of SCIM™(RR)=323 for the CAD subject considered in Example 5, below (see also FIG. 8). A comparison of this SCIM™(RR) value with that from Example 3 indicates that the method of the present invention allows one to discriminate between ischemia-induced hystereses in different subjects within a group which is sub-threshold for conventional ischemia detecting methods and techniques. A significant difference between the SCIM™(RR) values of the present example and with that of Example 3 which is equal to 125 indicates that the method possesses a good resolution even within a conventionally sub-threshold range of ischemic events. It also indicates that the method allows one to quantitatively differentiate the hystereses of the two subjects.

### EXAMPLE 5

### Hysteresis Curve for a Subject with ST Segment Depression Observed During Exercise Test

The test was carried out on a 61-year-old male subject using the apparatus and procedure described in Example 1 above. The subject exercised on a treadmill according to a quasi-stationary 20-minute protocol with a gradually increasing and gradually decreasing exercise load. A ST depression, indicating cardiac ischemia, was detected by the ROZINN system during the test. Independently, the patient also had a positive thallium ischemia stress test result. Panel A1 in FIG. 8 displays cardiac cycle length (RR interval) data set {T_{RR}(tⱼ)} versus time, tⱼ}, during the exercise test. A beat sampling rate with which the waveform analyzer sampled RR intervals was equal to 15 samples per minute. The data were obtained using left precordial V4 leads. Panel A2 in FIG. 8 displays filtered and smoothed data from the respective panels A1 as a functional dependence, <T_{RR}>=F(t). The smoothing procedure includes moving averaging with a two minute long moving window and is completed by the data fitting with the third-order polynomial functions within each two-minute long moving window. Panel B1 in FIG. 8 displays the raw RR interval fluctuations, δT_{RR}=T_{RR}-<T_{RR}>, versus time, t, during the exercise test. Panel B2 in FIG. 8 displays a dependence of STD, σ_{RR}=101(t), representing filtered and smoothed characteristics of the fluctuation magnitude from panel B1. Panel C1 in FIG. 8 displays the raw fluctuations, δT_{RR}, versus raw cardiac cycle length, T_{RR}, during exercise test. Finally, panel C2 in FIG. 8 displays a hysteresis loop parametrically represented by the dependencies <T_{RR}>=F(t) and σ_{RR}=Φ(t) plotted in panels A2 and B2, respectively. Notice a vertical line in panel C2 that closes the hysteresis loop and corresponds to step 8 in FIG. 4. The area of the closed hysteresis loop is relatively large as compared with those shown in FIGS. 6 and 7 and discussed in Examples 3 and 4. Appropriate renormalization (see Example 7) of the area results in the RR fluctuation ischemia index SCIM™ (RR)=323. This value is significantly greater than the values of 62 and 187 obtained in Examples 3 and 4 above. All the above cases demonstrate that the method of the present invention allows one to discriminate and quantitatively characterize the difference between (1) the levels of ischemia that can be detected by the conventional ST depression method and (2) the low levels of ischemia (illustrated in FIGS. 6 and 7) that are sub-threshold for the conventional method, which, therefore, can neither detect nor resolve such levels of ischemia or perfusion abnormality.

### EXAMPLE 6

### Fluctuation Analysis Method: An Example of Algorithm for Determining the RR Interval Fluctuations and Providing Their Characterization by the Moving STD

Let {(tₖ,Tₖ): k=1,2, . . . N} be a set of data points (the time instants {tₖ} are equidistant, tₖ-tₖ₋ₗ=const) obtained in the quasi-stationary exercise test as exemplified in panels A1 of FIGS. 6 through 8. The value of N in the above examples was about 400 and slightly varied from case to case. The data processing is essentially the same for the data collected by a Heart Rate Monitor, or by a regular Holter recorder and subsequently the waveform analyzer. The set {Tₖ} is a shorthand notation for the RR-interval data set {T^{k}_{RR}} or an equivalent cardiac cycle length data set. We define a k-th time m-window as a set of 2m+1 points {(tⱼ,Tⱼ): j=k−m, k−m+1, . . . ,k+m} that include and surround the point (tₖ,Tₖ). This in fact must be done for a variety of m-values among which one particular value is chosen as the final algorithmic step described below. Let us denote by ƒₖ(t) a quadratic or linear polynomial obtained by a linear regression such that (t, ƒₖ(t)) provides the best fit for the data points {tⱼ,Tⱼ} within the window. The function also describes the slow trend inside the window. The set of corresponding fluctuations {Tⱼ-ƒₖ(tⱼ)} is characterized by the standard deviation within the m-window given by the equation

$${σ}_{k}^{m} = \sqrt{\frac{1}{2 m} {\sum }_{j = k - m}^{j = k + m} {[ {T}_{j} - {f}_{k} ( {t}_{j} ) ]}^{2}} \qquad ( E .6 .1 )$$

This is repeated for various values of m. Then the optimal value of m is found from the requirement that σₖ^{m} does not include a trend, which reduces, in fact, to the requirement that the variation of σₖ^{m}/\sqrt{m} is minimum as a function of m.

$${σ}_{k} = {min}_{m , m > {m}_{min}} \{ \frac{{σ}_{k}^{m}}{\sqrt{m}} \} \qquad ( E .6 .2 )$$

The lower bound of m-values is determined by the requirement that all the calculations are robust and stable. We have chosen the value of mₘᵢₙ to correspond to a 45 second long time window. Thus, the optimized value of σₖ is taken as the current measure of fluctuations for the given, kth window. Having evaluated such optimal standard deviation for the RR intervals within the k-th, time window via optimized equation (E.6.2), we shift the window one time-step further and proceed to evaluation of σₖ₊₁. Since N is the total size of the sample (number of data points), this procedure is performed N−2m times and produces N−2m values of σₖ; the respective slow trend values ƒₖ(tₖ).

### EXAMPLE 7

### Calculation of a Quantitative Indicium of Cardiovascular Health

This example was carried out with the data obtained in Examples 3-5 above. FIG. 9 illustrates a comparative cardiovascular health analysis based on ischemia assessment by the method of the present invention. In this example an indicium of cardiovascular health (here designated the cardiac ischemia index (denoted by the acronym, SCIM™ (RR)) was designed, which was defined as a renornnalized area, S, of a quasi-stationary hysteresis loop on the plane RR interval fluctuation versus mean RR interval. The renormalization is done by dividing the loop area, S, by the product [(T_{RR})ₘₐₓ-(T_{RR})ₘᵢₙ)][(δT_{RR})ₘₐₓ-(δT_{RR})ₘᵢₙ]. For each particular subject this factor provides a correction for individual differences in the ranges of RR intervals occurring during the tests under the quasi-stationary treadmill exercise protocol. Broad variations of the values of SCIM™ (RR) across different subjects far exceed experimental error and indicate that the method of the present invention allows one to resolve and quantitatively characterize different levels of cardiac and cardiovascular health in a region in which the conventional ST depression method is sub-threshold and is unable to detect any exercise-induced ischemia. Thus, unlike a rough conventional ST-segment depression ischemic evaluation, the method of the present invention offers much more accurate assessing and monitoring of small variations of cardiac ischemia and associated changes of cardiac or cardiovascular health.

### EXAMPLE 8

### Illustration of Rapid Sympatho-Adrenal Transients

FIG. 9 illustrates a typical rapid sympathetic/parasympathetic nervous and hormonal adjustment of the RR (panels A and B) intervals to an abrupt stop after 10 minutes of exercise with increasing exercise load. Both panels depict temporal variations of the RR interval obtained from the right precordial lead V3 of the 12-lead multi-lead electrocardiogram. A sampling rate with which a waveform analyzer determined RR intervals was equal to 15 samples per minute. A human subject (a 47 years-old male) was at rest the first 10 minutes and then began to exercise with gradually increasing (during 10 minutes) exercise load (the portion of panels A to the left from the RR minimum). Then at the peak of the exercise load and the heart rate about 120 beat/min the subject stepped off the treadmill in order to initialize the fastest RR interval's adaptation to a complete abrupt stop of the exercise load. The subject rested sufficiently enough (13 minutes) in order to insure that the RR interval length had transitioned to a stationary, post-exercise average value. Panel B shows a blow up of the transitioning stage immediately after the abrupt stop of the exercise. The panel also includes the curve representing a data fit by a single exponential function:
<T_{RR}(t)>=(T_{RR})_{steady}[1−exp(−λ(t−tₘᵢₙ)] (E.8.1)
The recovery exponent, 1/λ=1/ln(5)=0.62 min⁻¹, corresponds to the observed recovery rate of about 0.15 s/min while the RR interval duration grows from 0.45 s to 0.6 s. Based on the above-described experiment, a definition for “rapid sympatho-adrenal and hormonal transients” or “rapid autonomic nervous system and hormonal transients” may be given.

Rapid transients due to autonomic nervous system and hormonal control refer to the transients with the RR interval transition rate of 0.15 s/min, which corresponds to the heart rate's rate of change of about 25 beat/min or faster rates of change in RR interval duration in response to a significant abrupt change (stop, reduction or increase) in exercise load (or other cardiac stimulus). The significant abrupt changes in exercise load are defined here as the load variations, which cause rapid variations in RR interval, comparable in size with the entire range from the exercise peak to the stationary average rest values.

### EXAMPLE 9

### Illustration of a Quasi-Stationary Exercise Protocol

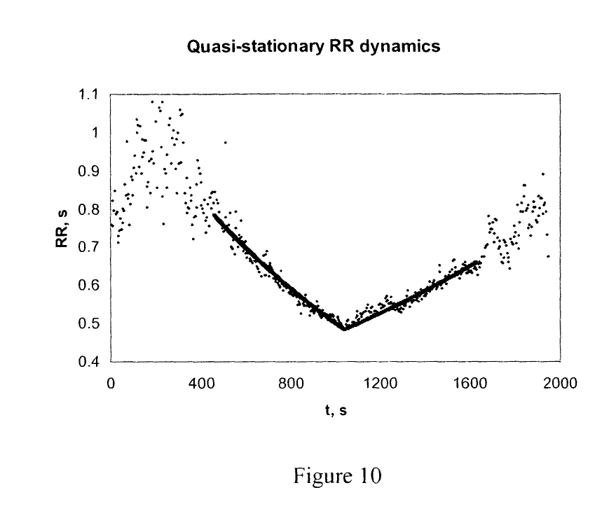

FIG. 10 illustrates a typical slow (quasi-stationary) RR interval adjustment measured during gradually increasing and gradually decreasing exercise load in a right pre-cordial V3 lead of the 12 lead electrocardiogram recording. The sampling was 15 QT and RR intervals per minute. A male, 47 year old subject exercised during two consecutive 10 minute long stages of gradually increasing and gradually decreasing exercise load. Both QT and RR intervals gradually approached the minimal values at about a peak exercise load (peak heart rate ˜120 beat/min) and then gradually returned to levels that were slightly lower than their initial pre-exercise rest values. The evolution of RR interval duration was well approximated by exponential fitting curves shown in gray. The ranges for the RR interval, there-and-back, time variations were 0.79 s-0.47 s-0.67 s (an average rate of change ˜0.032 s/min or ˜6 beat/min). The standard root-mean-square deviation, σ, of the observed RR interval lengths, shown by black dots, from their exponential fits were of an order of magnitude smaller than the average difference between the corresponding peak and rest values during the entire test (σ˜0.03 s). According to FIG. 10 such small perturbations, when associated with abrupt heart rate changes due to physiological fluctuations or due to discontinuities in an exercise load, may develop and decay faster than in 10 s, the time that is 60 times shorter than the duration of one gradual (ascending or descending) stage of the exercise protocol. Thus, because the evolution of the average values of RR interval occurs quite slowly under the quasi-stationary exercise protocol as compared with fast transients due to sympathetic/parasympathetic and hormonal control, the hysteresis loop practically does not depend on the peculiarities of the transients.

Based on the above-described experiment a definition for a gradual, or “quasi-stationary” exercise (or stimulation) protocol, can be quantitatively specified: A quasi-stationary exercise (or stimulation) protocol refers to two contiguous stages (each stage 3, 5, 8 or 10 minutes or longer in duration) of gradually increasing and gradually decreasing exercise loads or stimulation, such as:
1. Each stage's duration is approximately an order of magnitude (e.g., at least about ten times) longer than the average duration (˜1 minute) of a heart rate adjustment during an abrupt stop of the exercise between average peak load rate (˜120-150 beat/min) and average rest (˜50-70 beat/min) heart rate values.
2. The standard root-mean-square deviations of the original RR interval data set from their smooth and monotonic (for each stage) fits are of an order of magnitude (e.g., at least about ten times) smaller than the average differences between peak and rest RR interval values measured during the entire exercise under the quasi-stationary protocol.

As shown above (FIG. 10) a gradual quasi-stationary protocol itself allows one to substantially eliminate abrupt time dependent fluctuations from a measured RR interval data set because these fluctuations have short durations and small amplitudes. Their effect can be even further reduced by fitting each RR interval data set for each stage with a monotonic function of time. As a result the fitted RR interval values during each exercise stage can be presented as a substantially monotonic and smooth function of time. A similar conclusion can be drawn for the time course of the averaged fluctuations represented by the moving STD. Presented on the (<T_{RR}>,σ_{RR})-plane, these smooth dependencies give rise to a hysteresis loop, whose shape, area and other measures are quite similar to the hysteresis loops presented in FIGS. 6-8. Such hysteresis loops can provide an excellent measure of gradual ischemic exercise dependent changes in cardiac electrical conduction and can reflect cardiac health itself and cardiovascular system health in general.

The foregoing examples are illustrative of the present invention and are not to be construed as limiting thereof. The invention is defined by the following claims, with equivalents of the claims to be included therein.

1. A method of assessing cardiac ischemia in a subject to provide a measure of cardiovascular health in that subject, comprising said steps of:
(a) collecting a first RR-interval data set from said subject during a stage of gradually increasing heart rate;
(b) collecting a second RR-interval data set from said subject during a stage of gradually decreasing heart rate;
(c) separating fluctuations from a slow trend in said first RR-interval data set;
(d) separating fluctuations from a slow trend in said second RR-interval data set;
(e) comparing said fluctuations of said first RR-interval data set to said fluctuations of said second RR-interval data set to determine a difference between said fluctuation data sets; and
(f) generating from said comparison of step (e) a measure of cardiac ischemia during stimulation in said subject, wherein a greater difference between said first and second data sets indicates greater cardiac ischemia and lesser cardiac or cardiovascular health in said subject.

2. The method of claim 1, wherein:
said step (c) of separating fluctuations from at least one slow trend in said first RR-interval data set includes smoothing said first RR-interval data set to determine at least one slow trend in said first RR-interval data set; and
said step (c) of separating fluctuations from at least one slow trend in said second RR-interval data set includes smoothing said second RR-interval data set to determine at least one slow trend in said second RR-interval data set.

3. The method of claim 1, wherein said comparing step (e) is carried out at substantially equal trend values of said RR-intervals.

4. The method of claim 1, wherein said first and second RR-interval data sets are collected without an intervening rest stage.

5. The method of claim 1, wherein said first and second RR-interval data sets are collected under quasi-stationary conditions.

6. The method of claim 1, wherein said stage of gradually increasing heart rate and said stage of gradually decreasing heart rate are each at least 3 minutes in duration.

7. The method of claim 1, wherein said stage of gradually increasing heart rate and said stage of gradually decreasing heart rate are together carried out for a total time of from 6 minutes to 40 minutes.

8. The method of claim 1, wherein:
both said stage of gradually increasing heart rate and said stage of gradually decreasing heart rate are carried out between a peak rate and a minimum rate; and
said peak rates of both said stage of gradually increasing heart rate and said stage of gradually decreasing heart rate are the same.

9. The method of claim 8, wherein:
said minimum rates of both said stage of gradually increasing heart rate and said stage of gradually decreasing heart rate are substantially the same.

10. The method of claim 1, wherein said stage of gradually decreasing heart rate is carried out at at least three different heart-rate stimulation levels.

11. The method of claim 10, wherein said stage of gradually increasing heart rate is carried out at at least three different heart-rate stimulation levels.

12. The method of claim 1, wherein said stage of gradually increasing heart rate and said stage of gradually decreasing heart rate are carried out sequentially in time.

13. The method of claim 1, wherein said stage of gradually increasing heart rate and said stage of gradually decreasing heart rate are carried out separately in time.

14. The method of claim 1, wherein said heart rate during said stage of gradually increasing heart rate does not exceed more than 120 beats per minute.

15. The method of claim 1, wherein said heart rate during said stage of gradually increasing heart rate exceeds 120 beats per minute.

16. The method of claim 1, wherein said first and second RR-interval data sets are collected by pulse or blood pressure monitoring.

17. The method of claim 1, wherein said comparing step is preceded by the step of generating fluctuation curves for each of said data sets.

18. The method of claim 17, wherein said comparing step includes comparing the shapes of said fluctuation curves of each of said data sets.

19. The method of claim 17, wherein said comparing step includes determining a measure of the domain between said fluctuation curves.

20. The method of claim 19, wherein said comparing step includes a step of connecting said curves with a connecting segment to form a closed domain bounded by said fluctuation curves and said connecting segment.

21. The method of claim 20, wherein said comparing step includes determining a measure of the domain bounded by said fluctuation curves and said connecting segment.

22. The method of claim 17, wherein said comparing step includes both comparing the shapes of said fluctuation curves and determining a measure of the domain between said fluctuation curves.

23. The method of claim 17, further comprising the step of displaying said fluctuation curves.

24. The method of claim 1, wherein said separating step (c), said separating step (d) and said comparing step (e) are carried out by:
(i) smoothing said first and second RR-interval data sets to generate first and second slow trend data sets;
(ii) separating fluctuations from said second said slow trend in said first and second data sets to generate first and second fluctuation data sets;
(iii) generating a first fluctuations versus trend curve from said first slow trend data set and said first fluctuation data set;
(iv) generating a second fluctuations versus trend curve from said second slow trend data set and said second fluctuation data set;
(v) generating a hysteresis loop from said first fluctuations verses trend curve and said second fluctuations versus trend curve; and
(vi) determining a measure of the domain inside said smoothed hysteresis loop to thereby quantify a difference between said fluctuation data sets.

25. The method of claim 25, further comprising the step of:
adding a connecting segment between said first and second fluctuations versus trend curve to generate a closed hysteresis loop bounded by said first and second fluctuations versus trend curves and said connecting segment;
and wherein said determining step is carried out by determining a measure of the domain inside said smoothed closed hysteresis loop.

26. The method of claim 1, wherein said separating step (c), said separating step (d), and said comparing step (e) are carried out by:
(i) smoothing said first and second RR-interval data sets;
(ii) generating first and second smoothed trend versus time curves from said smoothed first and second RR-interval data sets;
(iii) generating first and second cardiac cycle length fluctuations versus time curves by separating fluctuations from slow trends;
(iv) generating an open hysteresis loop having two branches from said first and second trend versus time curves and said first and second fluctuations versus time curves
(v) connecting said branches of said open hysteresis loop to generate a closed hysteresis loop; and then
(vi) determining a measure of the domain inside said closed hysteresis loop to thereby quantify a difference between said fluctuation data sets.

27. The method of claim 26, wherein said generating step (iii) is followed by the step of fitting said first and second smoothed curves trend versus time curves.

28. The method of claim 26, wherein said generating step (iv) is followed by the step of smoothing said first and second fluctuation versus time curves.

29. The method of claim 1, further comprising the step of:
(g) comparing said measure of cardiac ischemia to at least one reference value; and then
(h) generating from said comparison of step (e) a quantitative indicium of cardiac ischemia for said subject.

30. The method of claim 29, further comprising the steps of:
(i) treating said subject with a cardiovascular therapy; and then
(j) repeating steps (a) through (f) to assess the efficacy of said cardiovascular therapy, in which a decrease in the quantitative indicium from before said therapy to after said therapy indicates an improvement in cardiac health in said subject from said cardiovascular therapy.

31. The method of claim 30, wherein said cardiovascular therapy is selected from the group consisting of aerobic exercise, muscle strength building, change in diet, nutritional supplement, weight loss, stress reduction, smoking cessation, pharmaceutical treatment, surgical treatment, and combinations thereof.

32. A computer system for assessing cardiac ischemia in a subject to provide a measure of cardiac or cardiovascular health in that subject, said system comprising:
(a) means for providing a first RR-interval data set collected from said subject during a stage of gradually increasing heart rate;
(b) means for providing a second RR-interval data set from said subject during a stage of gradually decreasing heart rate;
(c) means for separating fluctuations from slow trends in said first RR-interval data set;
(d) means for separating fluctuations from slow trends in said second RR-interval data set;
(e) means for comparing said fluctuations of said first RR-interval data set to said fluctuations of said second RR-interval data set at equal trend values of said RR-interval to determine said difference between said fluctuation data sets;
(f) means for generating from said comparison of step (e) a measure of cardiac ischemia during stimulation in said subject, wherein a greater difference between said first and second data sets indicates greater cardiac ischemia and lesser cardiac or cardiovascular health in said subject.

33. The system of claim 32, wherein said means (e) for comparing said fluctuations of said first RR-interval data set to said fluctuations of said second RR-interval data set compares said fluctuations at substantially equal trend values of said RR-interval.

34. The system of claim 32, further comprising:
(g) means for comparing said measure of cardiac ischemia to at least one reference value; and
(h) means for generating from said comparison of step (e) a quantitative indicium of cardiac ischemia for said subject.

35. A computer program product for assessing cardiac ischemia in a subject to provide a measure of cardiac or cardiovascular health in that subject, said computer program product comprising a computer usable storage medium having computer readable program code means embodied in said medium, said computer readable program code means comprising:
(a) computer readable program code for comparing a first RR-interval fluctuation data set to a second first RR-interval fluctuation data set to determine said difference between said data sets; and
(b) computer readable program code for generating from said code (a) a measure of cardiac ischemia during stimulation in said subject, wherein a greater difference between said first and second fluctuation data sets indicates greater cardiac ischemia and lesser cardiac or cardiovascular health in said subject.

36. The system of claim 35, wherein said computer readable program code for comparing a first RR-interval fluctuation data set to a second RR-interval fluctuation data set compares said fluctuations at substantially equal trend values of said RR-intervals.

37. The system of claim 35, further comprising:
(c) computer readable program code for comparing said measure of cardiac ischemia to at least one reference value; and then
(d) computer readable program code for generating from said code (e) a quantitative indicium of cardiac ischemia for said subject.
